(19) United States
(12) Patent Application Publication
(10) Pub. No.: US 2025/0352154 A1
(43) Pub. Date: Nov. 20, 2025

(54) ROTATION DRIVE APPARATUS AND CONTROL METHOD THEREOF, AND ROTATION EQUIPMENT

(71) Applicant: CANON KABUSHIKI KAISHA, Tokyo (JP)

(72) Inventors: TAKASHI SHIGEMORI, Kanagawa (JP); MASAKI FUJITA, Tokyo (JP); HITOSHI SUZUKI, Chiba (JP); TAKESHI YAMAMOTO, Kanagawa (JP)

(21) Appl. No.: 19/197,457

(22) Filed: May 2, 2025

(30) Foreign Application Priority Data
May 14, 2024 (JP) 2024-078798

(51) Int. Cl. A61B 6/03; A61B 6/00; H02N 15/00

(52) U.S. Cl. CPC A61B 6/035; A61B 6/4476; A61B 6/54; H02N 15/00

(57) ABSTRACT

A rotation drive apparatus includes: a rotor that includes a first magnetic force unit and is rotatable in a rotation direction around a rotation axis intersecting a direction of gravity; a stator that includes a second magnetic force unit; a support that supports the rotor positioned in a first position; and a control unit, wherein the first magnetic force unit is arranged circumferentially along the rotation direction on an outer peripheral side surface of the rotor, the second magnetic force unit is arranged in an arc shape along the rotation direction above the rotor, and, when the rotor is positioned at the first position and a levitation force with respect to the rotor is less than a predetermined value, the control unit rotates the rotor to a second position where the levitation force is equal to or greater than the predetermined value and levitates the rotor from the second position.

### BACKGROUND

### Field of the Disclosure

The present disclosure relates to a rotation drive apparatus and a control method thereof, and rotation equipment.

### Description of the Related Art

Gantries for computed tomography (CT), for example, are known as rotation equipment with a configuration in which a rotor is rotated in a contactless state by the action of magnetism. Furthermore, such a configuration is known to be applied to a magnetic levitation motor.

In a general X-ray CT apparatus, an X-ray tube is attached to the rotor side of a magnetic force unit that includes a stator and a rotor, and scanning is executed by rotating the rotor. In order to speed up rotation of the rotor, a magnetic levitation motor using a magnetic bearing is used for the bearing of the rotor to eliminate the sliding part of the motor. When an actuator for rotation and an actuator for magnetic levitation are individually arranged for rotation and magnetic levitation of the motor, the whole apparatus tends to be large. In order to realize the miniaturization of the apparatus by avoiding such an enlargement of the apparatus, a configuration in which the rotation and magnetic levitation of the motor are controlled by the same actuator is required.

The configuration described in Japanese Patent Application Laid-Open No. H07-184345 is known as one of the configurations in which the rotation torque and levitation force are generated by the same actuator in the magnetic levitation motor. The configuration described in Japanese Patent Application Laid-Open No. H07-184345 is a configuration that uses an induction motor, and both the rotation torque and the levitation force are acquired by applying a magnetic force from coils on the side of the stator to a rotor composed of a magnetic material.

In CT apparatuses, high positional accuracy is required when scanning during rotation. Therefore, synchronous motors are used for the CT apparatuses instead of induction motors. In the synchronous motor, permanent magnets are used in either the stator or the rotor. In addition, for high torque, coils in the synchronous motor are used with a core such as an iron core or the like. Therefore, an attractive force is always generated between the stator and the rotor.

When the rotor is levitated from the magnetic bearing, as described above, an attractive force generated between the stator and the rotor pulls the rotor in a direction different from the direction of gravity, causing the rotor to vibrate. As a result, it is difficult for the rotor to levitate stably when the stator transitions from a seated state supported by the magnetic bearing to a levitated state. In particular, since the CT apparatus is large, there is a concern that when the rotor vibrates, the rotor comes into contact with the magnetic bearing, causing the rotor or the magnetic bearing to wear out.

### SUMMARY

It is an object of the present disclosure to provide a rotation drive apparatus and a control method thereof, and rotation equipment that can levitate a rotor while avoiding enlargement of the whole apparatus.

According to one aspect of the present disclosure, there is provided a rotation drive apparatus including: a rotor that includes a first magnetic force unit and is rotatable in a rotation direction around a rotation axis intersecting a direction of gravity; a stator that includes a second magnetic force unit; a support that supports the rotor positioned in a first position; and a control unit, wherein the first magnetic force unit is arranged circumferentially along the rotation direction on an outer peripheral side surface of the rotor, wherein the second magnetic force unit is arranged in an arc shape along the rotation direction above the rotor, and wherein, when the rotor is positioned at the first position and a levitation force with respect to the rotor is less than a predetermined value, the control unit rotates the rotor to a second position where the levitation force is equal to or greater than the predetermined value and levitates the rotor from the second position.

According to another aspect of the present disclosure, there is provided a rotation drive apparatus including: a rotor that includes a first magnetic force unit and is rotatable in a rotation direction around a rotation axis intersecting a direction of gravity; a stator that includes a second magnetic force unit; a support that supports the rotor positioned in a first position; and a control unit, wherein the first magnetic force unit is arranged circumferentially along the rotation direction on an outer peripheral side surface of the rotor, wherein the second magnetic force unit is arranged in an arc shape along the rotation direction above the rotor, and wherein, when the rotor is positioned at the first position and a cogging torque with respect to the rotor is equal to or more than a predetermined value, the control unit rotates the rotor to a second position where the cogging torque is less than the predetermined value and levitates the rotor from the second position.

Further features of the present disclosure will become apparent from the following description of exemplary embodiments with reference to the attached drawings.

### BRIEF DESCRIPTION OF THE DRAWINGS

FIG. 1A is a schematic diagram illustrating a rotation drive apparatus according to one or more aspects of the present disclosure.

FIG. 1B is a schematic diagram illustrating the rotation drive apparatus according to one or more aspects of the present disclosure.

FIG. 1C is a schematic diagram illustrating the rotation drive apparatus according to one or more aspects of the present disclosure.

FIG. 1D is a schematic diagram illustrating the rotation drive apparatus according to one or more aspects of the present disclosure.

FIG. 2A is a schematic diagram illustrating the arrangement of permanent magnets in the rotation drive apparatus according to one or more aspects of the present disclosure.

FIG. 2B is a schematic diagram illustrating the arrangement of coils in the rotation drive apparatus according to one or more aspects of the present disclosure.

FIG. 2C is a schematic diagram illustrating a thrust constant in the rotation drive apparatus according to one or more aspects of the present disclosure.

FIG. 2D is a schematic diagram illustrating the thrust constant in the rotation drive apparatus according to one or more aspects of the present disclosure.

FIG. 3A is a schematic diagram illustrating the arrangement of sensors in the rotation drive apparatus according to one or more aspects of the present disclosure.

FIG. 3B is a schematic diagram explaining a method of calculating the displacement of a rotor from the sensors in the rotation drive apparatus according to one or more aspects of the present disclosure.

FIG. 4 is a schematic diagram illustrating a current control system in the rotation drive apparatus according to one or more aspects of the present disclosure.

FIG. 5 is a block diagram illustrating a control loop for calculating the magnitude of a torque applied to the rotor in the rotation drive apparatus according to one or more aspects of the present disclosure.

FIG. 6 is a schematic diagram illustrating the torque controlled by the coils in the rotation drive apparatus according to one or more aspects of the present disclosure.

FIG. 7A is a schematic diagram illustrating an attractive force acting between the permanent magnet and the core of the coil in the rotation drive apparatus according to one or more aspects of the present disclosure.

FIG. 7B is a schematic diagram illustrating an attractive force acting between the permanent magnets and the cores of the coils in the rotation drive apparatus according to one or more aspects of the present disclosure.

FIG. 8A is a schematic diagram illustrating forces acting according to a position in a Y direction of the rotor in the rotation drive apparatus according to one or more aspects of the present disclosure.

FIG. 8B is a schematic diagram illustrating the forces acting according to a position in the Y direction of the rotor in the rotation drive apparatus according to one or more aspects of the present disclosure.

FIG. 8C is a schematic diagram illustrating the force acting according to a position in the Y direction of the rotor in a rotation drive apparatus according to one or more aspects of the present disclosure.

FIG. 9A is a schematic diagram illustrating the state in which the rotor comes into contact with a seating bearing in the rotation drive apparatus according to one or more aspects of the present disclosure.

FIG. 9B is a schematic diagram illustrating a state in which the rotor comes into contact with the seating bearing in the rotation drive apparatus according to one or more aspects of the present disclosure.

FIG. 10 is a schematic diagram illustrating the arrangement of the permanent magnet giving priority to a torque in the Y direction in the rotation drive apparatus according to one or more aspects of the present disclosure.

FIG. 11A is a schematic diagram illustrating a state of the rotor that achieves an electric angle that facilitates stable levitation in the rotation drive apparatus according to one or more aspects of the present disclosure.

FIG. 11B is a schematic diagram illustrating a state of the rotor that achieves an electric angle that facilitates stable levitation in the rotation drive apparatus according to one or more aspects of the present disclosure.

FIG. 12A is an example of data showing the transition of the position of the rotor in an X direction during stable levitation in the rotation drive apparatus according to one or more aspects of the present disclosure.

FIG. 12B is an example of data showing the transition of the position of the rotor in the X direction during stable levitation in the rotation drive apparatus according to one or more aspects of the present disclosure.

FIG. 13A is an example of data showing the transition of a current command value during stable levitation in the rotation drive apparatus according to one or more aspects of the present disclosure.

FIG. 13B is an example of data showing the transition of the current command value during stable levitation in the rotation drive apparatus according to one or more aspects of the present disclosure.

FIG. 14 is a flowchart illustrating a levitation procedure of the rotor by a levitation procedure executor in the rotation drive apparatus before the application of the present disclosure.

FIG. 15 is a flowchart illustrating a levitation procedure of the rotor by the levitation procedure executor when a procedure for rotating the rotor is added before the levitation procedure is executed in the rotation drive apparatus according to one or more aspects of the present disclosure.

FIG. 16A is a schematic diagram illustrating a state of a rotor that results in an electric angle with a large cogging torque in a rotation drive apparatus according to one or more aspects of the present disclosure.

FIG. 16B is a schematic diagram illustrating a state of the rotor that results in an electric angle with a large cogging torque in the rotation drive apparatus according to one or more aspects of the present disclosure.

FIG. 17 is a flowchart illustrating a levitation procedure of the rotor by the levitation procedure executor in the rotation drive apparatus according to one or more aspects of the present disclosure.

FIG. 18 is a schematic diagram illustrating rotation equipment according to one or more aspects of the present disclosure.

### DESCRIPTION OF THE EMBODIMENTS

### First Embodiment

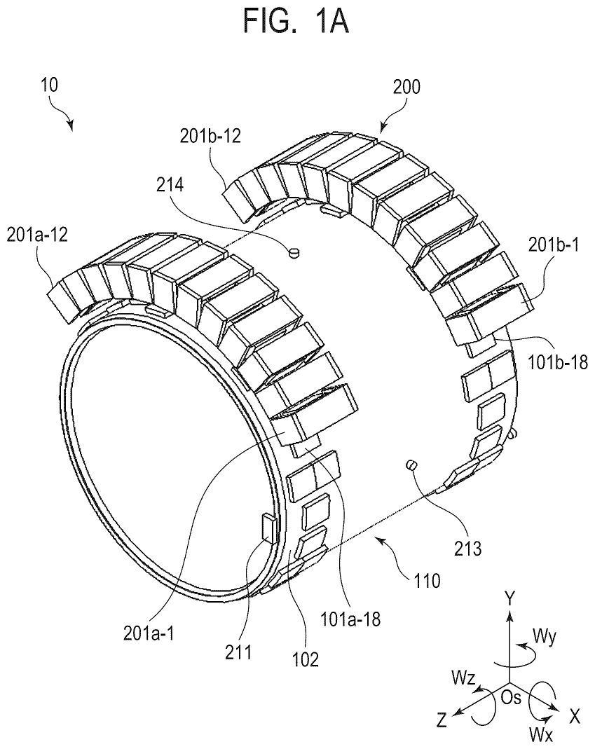

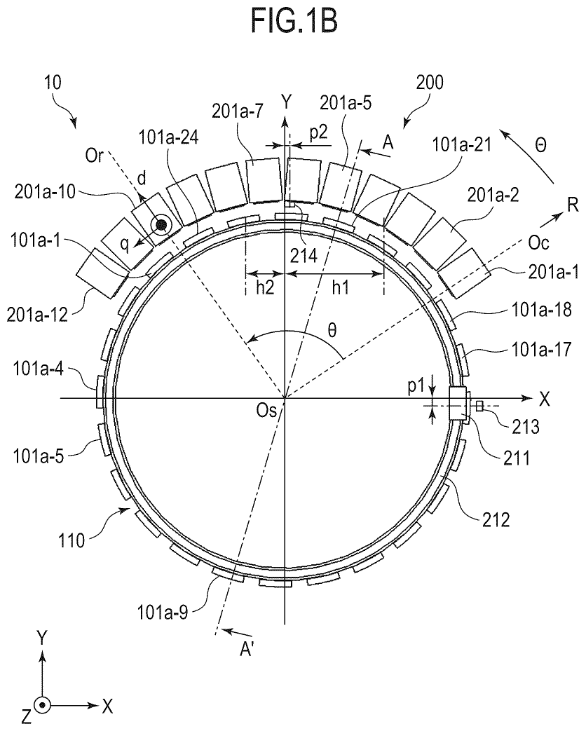

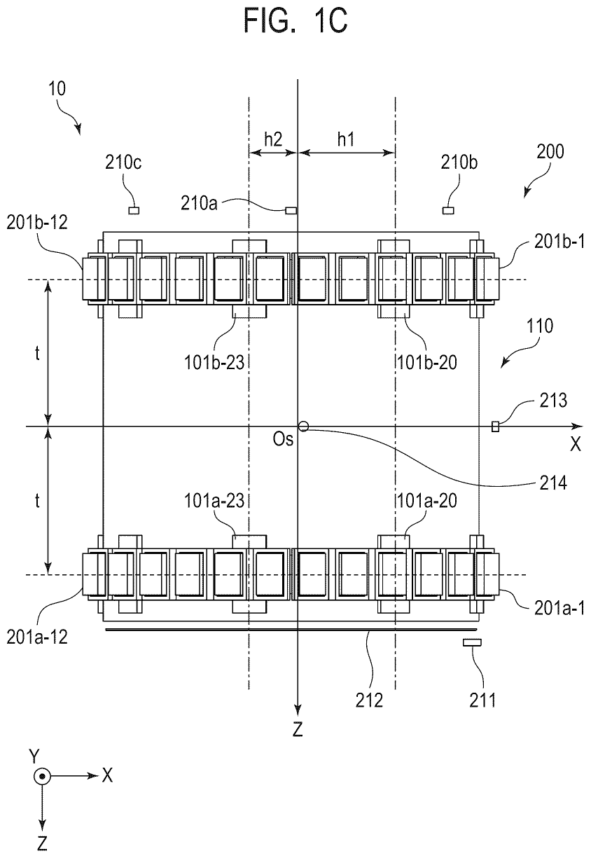

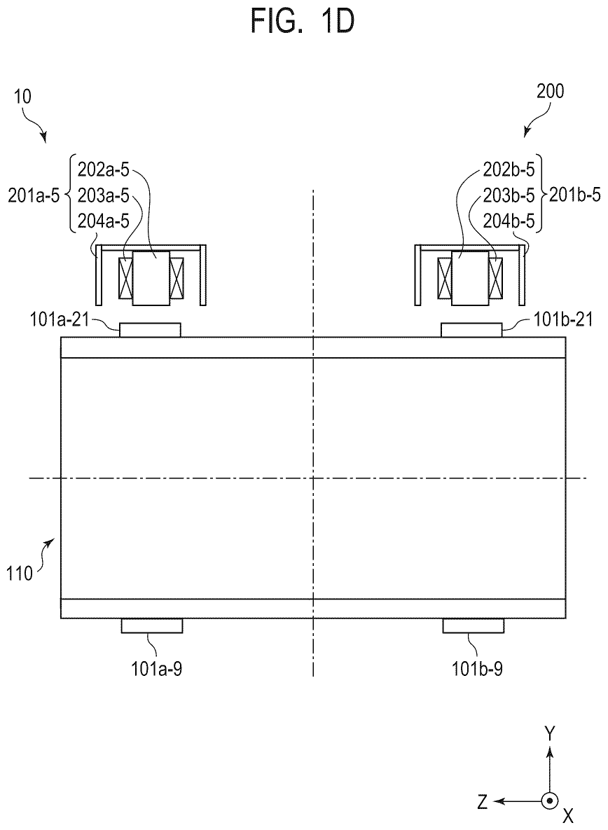

A rotation drive apparatus according to a first embodiment of the present disclosure will be described with reference to FIG. 1A to FIG. 15. The rotation drive apparatus according to the present embodiment includes a rotor having a rotation axis intersecting the direction of gravity and a stator arranged on the upper side of the rotor in the direction of gravity, and rotates the rotor around the axis in a contactless state while magnetically levitating the rotor in the direction of gravity.

Here, the coordinate axes directions, and the like used in the following description are defined. First, the rotation axis around which the rotor 110 described later rotates is defined as a Z-axis. Further, an XY-plane is taken so as to be orthogonal to the Z-axis, and two axes orthogonal to each other in the XY-plane are defined as an X-axis and a Y-axis, respectively. Further, the direction along the X-axis is defined as an X direction, the direction along the Y-axis is defined as a Y direction, and the direction along the Z-axis is defined as a Z direction. Further, the rotation direction around the X-axis is defined as a Wx direction, the rotation direction around the Y-axis is defined as a Wy direction, and the rotation direction around the Z-axis is defined as a Wz direction. The origin of the XYZ coordinates including the X-axis, the Y-axis, and the Z-axis is set as Os. The origin Os is the position where the center lines of the coils arranged in the stator 200 described later are connected. The positive direction of each rotation in the Wx direction, the Wy direction and the Wz direction is a clockwise direction toward the positive direction of each axis in which each the X-axis, the Y-axis and the Z-axis extends from the origin Os. Further, an R-axis is taken in a radial direction in which a radius increases in a plane along the XY-plane with the Z-axis as the central axis. Further, in the following description, the Z direction is taken as the horizontal direction and the Y direction as the direction of gravity. Note that the X direction, the Y direction and the Z direction are not necessarily limited to directions orthogonal to each other, but can be defined as directions intersecting each other. Also note that the Z direction does not necessarily have to be the horizontal direction, but can be a direction inclined with respect to the horizontal direction. In this case, the X direction and the Z direction can be defined in the same way with respect to the Z direction.

First, the configuration of the rotation drive apparatus according to the present embodiment will be described with reference to FIG. 1A to FIG. 1D. FIG. 1A is a perspective view illustrating the rotation drive apparatus 10 according to the present embodiment. FIG. 1B is a side view of the rotation drive apparatus 10 according to the present embodiment viewed in the Z direction. FIG. 1C is a top view of the rotation drive apparatus 10 according to the present embodiment viewed in the Y direction. FIG. 1D is a cross-sectional view taken along a line A-A′ illustrated in FIG. 1B viewed in the X direction.

As illustrated in FIG. 1A to FIG. 1C, the rotation drive apparatus 10 according to the present embodiment includes a rotor 110 and a stator 200. The rotor 110 includes a plurality of first permanent magnets 101a and a plurality of second permanent magnets 101b as permanent magnet groups. The stator 200 includes a plurality of first coils 201a and a plurality of second coils 201b as coil groups. The rotation drive apparatus 10 includes a current control system 300 described later as a control unit that controls the drive of the rotor 110.

Note that, in the following description, each permanent magnet in the plurality of first permanent magnets 101a is simply denoted as “first permanent magnet 101a” and each permanent magnet in the plurality of second permanent magnets 101b is simply denoted as “second permanent magnet 101b,” unless otherwise necessary to be specifically distinguished. When it is necessary to identify each permanent magnet individually, Expressions such as “first permanent magnet 101a-1”, “first permanent magnet 101a-2”, “second permanent magnet 101b-1”, “second permanent magnet 101b-2”, and the like are used, with numeral signs each further having a branch number following “-” to identify each permanent magnet individually. In addition, “first permanent magnet 101a” and “second permanent magnet 101b” may be collectively referred to simply as “permanent magnet 101” without distinction.

Similarly, unless otherwise necessary to be specifically distinguished, each coil in the plurality of first coils 201a is simply denoted as “first coil 201a” and each coil in the plurality of second coils 201b is simply denoted as “second coil 201b”. When it is necessary to identify each coil individually, Expressions such as “first coil 201a-1”, “first coil 201a-2”, “second coil 201b-1”, “second coil 201b-2”, and the like, are used, with numeral signs each further having a branch number following “-” to identify each coil individually. The components of the coil are also denoted each with a numeral sign having a branch number in the same manner to be identified individually as necessary. For the coil group of the plurality of first coils 201a or the plurality of second coils 201b, a symbol “j” as an index of the coil is used as described later. Note that “j” is a positive integer. Also, “first coil 201a” and “second coil 201b” may be collectively referred to simply as “coil 201” without distinction.

As illustrated in FIG. 1A, the rotor 110 has a hollow cylindrical shape with the Z axis as its central axis. The rotor 110 is configured to be rotatable in the Wz direction, which is a rotation direction with the Z-axis as a rotation axis centered. Note that the shape of the rotor 110 is not limited to the hollow cylindrical shape. The shape of the rotor 110 may be a shape that can be rotatable around a rotation axis along a direction intersecting the direction of gravity, and may be another shape such as a cylindrical shape depending on the configuration of equipment that utilizes the rotation of the rotor 110, and the like.

The plurality of first permanent magnets 101a, which are a group of permanent magnets, are mounted and installed on the outer peripheral side surface of the rotor 110 so as to be uniformly arranged in one line in a circumferential shape along the Wz direction, which is the rotation direction. The plurality of second permanent magnets 101b, which are a group of permanent magnets, are also mounted and installed on the outer peripheral side surface of the rotor 110 so as to be uniformly arranged in one line in a circumferential shape along the Wz direction. For example, the plurality of first permanent magnets 101a are circumferentially arranged on the outer peripheral side of one end in the Z direction of the rotor 110, and the plurality of second permanent magnets 101b are circumferentially arranged on the outer peripheral side of the other end in the Z direction of the rotor 110. Thus, the plurality of first permanent magnets 101a and the plurality of second permanent magnets 101b are arranged in the rotor 110. The first permanent magnets 101a and the second permanent magnets 101b function as first magnetic force units between which and the first coil 201a and the second coil 201b of the stator 200 magnetic forces act, respectively.

The plurality of first coils 201a and the plurality of second coils 201b are arranged on the stator 200 so that the plurality of first coils 201a and the plurality of second coils 201b are positioned to face the first permanent magnets 101a and the second permanent magnets 101b above the rotor 110 in the Y direction outside the rotor 110. The plurality of first coils 201a are mounted in positions where they can face the plurality of first permanent magnets 101a of the rotor 110 so as to be evenly arranged in one line in an arc shape along the Wz direction. The plurality of second coils 201b are mounted in positions where they can face the plurality of second permanent magnets 101b of the rotor 110 so as to be evenly arranged in one line in an arc shape along the Wz direction. Thus, the plurality of first coils 201a and the plurality of second coils 201b are arranged in the stator 200. The first coils 201a and the second coils 201b function as a second magnetic force unit between which and the first permanent magnet 101a and the second permanent magnet 101b of the rotor 110 magnetic forces act, respectively.

The set of the first coils 201a and the first permanent magnets 101a, and the set of the second coils 201b and the second permanent magnets 101b are arranged on the stator 200 and the rotor 110 symmetrically with the XY-plane as the plane of symmetry. Thus, the rotor 110 is arranged on the lower side of the plurality of first coils 201a and the plurality of second coils 201b in the direction of gravity, and rotates around the Z-axis by rotation control while levitating in the Y direction by levitation control.

In the present embodiment, the first coils 201a-1 to 201a-12 are arranged in order in a Θ direction, which is the circumferential direction along the Wz direction, with a reference Oc, which is a straight line extending obliquely upward along the XY-plane from the origin Os, as the reference of the stator 200. Similarly, the second coils 201b-1 to 201b-12 are arranged in order in the @ direction with the reference Oc as the reference of the stator 200. Further, the first permanent magnets 101a-1 to 101a-24 are arranged in order in the Θ direction with a reference Or, which is a straight line extending from the origin Os toward a predetermined position on the outer peripheral side surface of the rotor 110, as the reference of the rotor 110. Similarly, the second permanent magnets 101b-1 to 101b-24 are arranged in order in the Θ direction with the reference Or as the reference of the rotor 110.

Note that the number of the first coils 201a, the number of the second coils 201b, the number of the first permanent magnets 101a, and the number of the second permanent magnets 101b are not limited to those illustrated in the present embodiment. The number of the first coils 201a, the number of the second coils 201b, the number of the first permanent magnets 101a, and the number of the second permanent magnets 101b can be suitably changed according to thrusts required in a q-axis direction, a d-axis direction, and the Z-direction, and the like, which will be described later. Also note that, although the number of the first coils 201a and the number of the second coils 201b are equal to each other, they do not necessarily have to be equal to each other. Also note that, although the number of the first permanent magnets 101a and the number of the second permanent magnets 101b are equal to each other, they do not necessarily have to be equal to each other.

In the present embodiment, a case will be described in which lines of sets of the permanent magnets and the coils facing each other are two lines including the line of the set of the first permanent magnets 101a and the first coils 201a and the line of the set of the second permanent magnets 101b and the second coils 201b. However, the lines of the set of the permanent magnets and the coils are not limited to this case. The line of the set of the permanent magnets and the coils may be one line, or may be not only two lines but also three or more lines in which lines of the same set as the set of the first permanent magnets 101a and the first set of coils 201a are further provided. Note that, when the line of the set of the permanent magnets and the coils is one line, it is desirable that the coils are arranged in plane symmetry with the YZ-plane as the plane of symmetry from the viewpoint of stably controlling the rotor 110.

FIG. 1B is a side view of FIG. 1A viewed from the Z direction. Hereinafter, the first coils 201a and the first permanent magnets 101a will be described with reference to FIG. 1B and FIG. 1C. Note that the second coil 201b has the same configuration as the first coil 201a, and the second permanent magnet 101b has the same configuration as the first permanent magnet 101a.

As illustrated in FIG. 1B, the plurality of first permanent magnets 101a are mounted and installed along the circumferential direction of the rotor 110 so as to line up on the outer peripheral side surface of the rotor 110. The rotor 110 has a yoke 102 installed on its outer peripheral side surface. The plurality of first permanent magnets 101a are attached to the outer peripheral side surface of the rotor 110 via the yoke 102. Thus, the yoke 102 is arranged on the rear side of the first permanent magnets 101a. The yoke 102 is made of a metal having magnetism, such as iron, SUS400 series stainless steel, or the like to increase the magnetic force of the first permanent magnet 101a. The yoke 102 itself has strength and may also serve as a base member of the rotor 110. The yoke 102 may be installed separately from or integrated with the base member of the rotor 110. Note that it is not necessary for the yoke 102 to have the rotor 110 installed and the yoke 102 may not be installed. Even in this case, it is possible to perform levitation control and rotation control of the rotor 110.

Here, the rotation angle, which is an angle in the Wz direction of the rotor 110, is set to θ. The rotation angle θ is an angle from the reference Oc in the Wz direction on the side of the stator 200 to the reference Or in the Wz direction on the side of the rotor 110. Note that the reference Oc in the Wz direction on the side of the stator 200 is a straight line connecting the first coil 201a-1 arranged in an arc shape and the origin Os. The reference Or in the Wz direction on the side of the rotor 110 is a straight line connecting the middle of the first permanent magnet 101a-1 and the first permanent magnet 101a-24 and the origin Os.

FIG. 1C is a top view of FIG. 1A viewed from the Y direction. Note that, in FIG. 1C, side yokes 204a and 204b described later are omitted to illustrate the arrangement of the first coils 201a and the second coils 201b for ease of explanation. As illustrated in FIG. 1C, the plurality of first coils 201a and the plurality of second coils 201b are mounted and installed in plane symmetry with the YZ-plane as the plane of symmetry in the stator 200. Further, the plurality of first coils 201a and the plurality of second coils 201b are mounted and arranged in plane symmetry with the XY-plane as the plane of symmetry in the stator 200.

Further, the stator 200 includes an X sensor 213, a Y sensor 214, Z sensors 210a, 210b, and 210c, and a Wz sensor 211 as sensors for detecting the displacement or the attitude of the rotor 110. The displacement of the rotor 110 in the X direction can be detected based on the detection value of the X sensor 213. The displacement of the rotor 110 in the Y direction can be detected based on the detection value of the Y sensor 214. The displacement in the Z direction, the displacement in the Wx direction, and the displacement in the Wy direction of the rotor 110 can be detected based on the detected values of the Z sensors 210a, 210b, and 210c. The displacement of the rotor 110 in the Wz direction can be detected based on the detected values of the Wz sensor 211. The detection of the angular displacement is performed by a position controller 301 described later.

The X sensor 213 detects the distance in the X direction between the rotor 110 and the X sensor 213. The Y sensor 214 detects the distance in the Y direction between the rotor 110 and the Y sensor 214. The Z sensor 210a detects the distance in the Z direction between the rotor 110 and the Z sensor 210a. The Z sensor 210b detects the distance in the Z direction between the rotor 110 and the Z sensor 210b. The Z sensor 210c detects the distance in the Z direction between the rotor 110 and the Z sensor 210c. The X sensor 213, Y sensor 214, and Z sensors 210a, 210b, and 210c are not limited to any particular sensor, but may be eddy current sensors, displacement sensors, or other sensors that can detect distance. Further, optical sensors, magnetic sensors, or the like may be selected as these sensors with a separate scale for detection.

The X sensor 213 is positioned laterally in the X direction with respect to the rotor 110 so as to face the outer peripheral side surface of the rotor 110. The Y sensor 214 is positioned upward in the Y direction with respect to the rotor 110 so as to face the outer peripheral side surface of the rotor 110. The Z sensors 210a, 210b, 210c are positioned laterally in the Z direction with respect to the rotor 110 so as to face the circumferential end surface of the rotor 110 in the Z direction.

The X sensor 213 and the Y sensor 214 are installed so as to perform detection at a position deviated from the vertex of the outer peripheral side surface of the rotor 110 in the Y direction or the X direction in order to perform detection of the outer peripheral side surface which is the curved surface portion of the rotor 110, respectively. This is due to the following reasons for the X sensor 213 and the Y sensor 214.

The reason for the X sensor 213 is as follows. When the rotor 110 is levitated and controlled, if the X sensor 213 is installed so as to detect the vertex of the outer peripheral side surface in the X direction, the X sensor 213 has a position where the X sensor 213 exhibits the same detection values on the positive side and the negative side in the Y direction when the rotor 110 is controlled to move in the Y direction. In order to avoid such the same detection values, as illustrated in FIG. 1B, the X sensor 213 is installed at a position offset by an offset amount p1 on the negative side in the Y direction from the position facing the vertex of the outer peripheral side surface of the rotor 110 in the X direction.

Further, the X sensor 213 is arranged so that the range of the levitation control in the Y direction of the rotor 110 is equal to or less than the offset amount p1. Since the X sensor 213 is arranged in this manner, the X sensor 213 does not exceed the vertex of the outer peripheral side surface of the rotor 110 in the X direction in terms of configuration or control in the Y direction, the X sensor 213 does not exhibit the same detection values on the positive side and the negative side in the Y direction.

Since the position of the X sensor 213 is offset in the Y direction from the position where the X sensor 213 faces the vertex of the outer peripheral side face of the rotor 110 in the X direction, the position of the rotor 110 can be accurately detected by the X sensor 213. Note that, although the X sensor 213 is offset in the negative side in the Y direction in FIG. 1B, the X sensor 213 may be offset in the positive side in the Y direction.

The reason for the Y sensor 214 is as follows. Similarly, when the rotor 110 is levitated and controlled, if the Y sensor 214 is installed so as to detect the vertex of the outer peripheral side surface in the Y direction, the Y sensor 214 has a position where the Y sensor 214 exhibits the same detection values on the positive side and the negative side in the X direction when the rotor 110 is controlled to move in the X direction. In order to avoid such the same detection values, as illustrated in FIG. 1B, the Y sensor 214 is installed at a position offset by an offset amount p2 on the positive side in the X direction from the position facing the vertex of the outer peripheral side surface of the rotor 110 in the Y direction.

Further, the Y sensor 214 is arranged so that the range of the movement control in the X direction of the rotor 110 is equal to or less than the offset amount p2. Since the Y sensor 214 is arranged in this manner, the Y sensor 214 does not exceed the vertex of the outer peripheral side surface of the rotor 110 in the Y direction in terms of configuration or control in the X direction, the Y sensor 214 does not exhibit the same detection value on the positive side and the negative side in the X direction.

Since the position of the Y sensor 214 is offset in the X direction from the position where the Y sensor 214 faces the vertex of the outer peripheral side face of the rotor 110 in the Y direction, the position of the rotor 110 can be accurately detected by the Y sensor 214. Note that, although the Y sensor 214 is offset in the positive side in the X direction in FIG. 1B, the Y sensor 214 may be offset in the negative side in the X direction.

However, when the position of the rotor 110 is simultaneously measured by the X sensor 213 and the Y sensor 214, the displacement of the rotor 110 can be detected. Therefore, it is not necessary that the X sensor 213 and the Y sensor 214 are offset as described above.

The Wz sensor 211 is positioned on the side opposite to the Z sensors 210a, 210b, and 210c in the Z direction with respect to the rotor 110 so as to face the circumferential end surface of the rotor 110 in the Z direction. A scale 212 is mounted and installed on the circumferential end surface of the rotor 110 which the Wz sensor 211 faces. The Wz sensor 211 detects the rotation angle of the rotor 110 in the Wz direction by reading a pattern on the scale 212. The Wz sensor 211 may be any sensor capable of detecting the angle of an optical type, a magnetic type, or the like, and may be of an absolute type or an increment type used with origin detection means.

FIG. 1D is a cross-sectional view illustrating the cross section along the line A-A′ illustrated in FIG. 1B. Here, the structure and mounting position of the first coil 201a and the second coil 201b will now be described with reference to FIG. 1D. The first coil 201a and the second coil 201b are each formed by winding a conductor wire around a magnetic material such as a silicon steel sheet or the like used in general motors.

Specifically, as illustrated in FIG. 1D, the first coil 201a-5 is formed by winding a winding 203a-5 around a core 202a-5 formed of a magnetic material such as a silicon steel sheet and attaching a U-shaped side yoke 204a-5 to the core 202a-5. The side yoke 204a-5, like the core 202a-5, is formed of a magnetic material such as a silicon steel sheet. The core 202a-5 is arranged so that its central axis faces the Z-axis along the XY-plane. The winding 203a-5 is wound around the core 202a-5 with the center axis of the core 202a-5 as the center. The side yoke 204-5 is composed of three plate-like parts covering the outer side in the R-axis direction and both sides in the Z-direction of the core 202a-5. All of the other first coils 201a have the same configuration as the first coils 201a-5.

Note that, although the configuration with the side yoke 204a attached is described as an example in the present embodiment, it is not limited to this configuration. The side yoke 204a only needs to be installed when a torque in the Z direction is required, and it is possible to perform levitation control and rotation control of the rotor 110 is possible even when the side yoke 204a is not installed.

As described above, since the first coil 201a is configured to include the core 202a and the side yoke 204a, the magnetoresistance is reduced, and the magnetic flux density generated by the first permanent magnet 101a and the magnetic flux density generated by current flowing through the winding 203a can be increased. Since each magnetic flux density can be increased, the torque generated in each direction can also be increased.

Note that the second coil 201b has the same configuration as the first coil 201a. The core 202b, the winding 203b and the side yoke 204b, which are components of the second coil 201b, correspond to the core 202a, the winding 203a and the side yoke 204a, respectively, which are components of the first coil 201a.

The plurality of first permanent magnets 101a and the plurality of second permanent magnets 101b are preferably arranged in plane symmetry in the rotor 110 with the XY-plane as the plane of symmetry, that is, with a plane orthogonal to the Z-axis, which is the rotation axis, as the plane of symmetry. Further, it is preferable that the weight of the rotor 110 is equal in the Z direction. Since the weight of the rotor 110 is equal, the inclination and eccentricity of the rotor 110 caused by the variation of the weight can be reduced, so that the control of the rotor 110 can be easily performed. It is preferable that both the first coils 201a and the second coils 201b are arranged in plane symmetry in the stator 200 with the XY-plane as the plane of symmetry, that is, with a plane orthogonal to the Z-axis which is the rotation axis as the plane of symmetry. Thus, by arranging the set of the first permanent magnets 101a and the first coils 201a and the set of the second permanent magnets 101b and the second coils 201b symmetrically in this way, the positions where the attractive forces are generated in the respective sets are located in plane symmetry with the XY-plane as the plane of symmetry. Since the positions where the attractive forces are generated are located symmetrically in this way, the inclination and eccentricity of the rotor 110 caused by variations in the positions can be reduced, and the control of the rotor 110 can be easily performed.

Further, the first coils 201a and the second coils 201b are preferably arranged in plane symmetry in the stator 200 with the YZ-plane as the plane of symmetry, that is, with a plane including the Z-axis which is the rotation axis and oriented along the Y direction which is the direction of gravity as the plane of symmetry. The symmetry of such an arrangement makes the positions where the attractive forces are generated symmetrical with the YZ plane as the plane of symmetry, which makes the control of the rotor 110 performed more easily.

Note that, although the first permanent magnets 101a and the second permanent magnets 101b, and the first coils 201a and the second coils 201b are arranged symmetrically in the above description, it is not necessary that they are arranged symmetrically. The inclination and eccentricity of the rotor 110 can be corrected by adjusting the gap between the coils and the permanent magnets and the amount of current flowing through the coils.

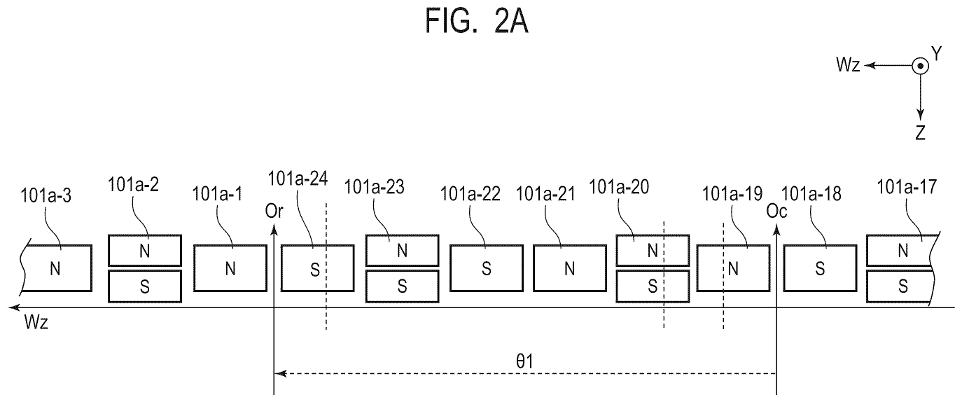

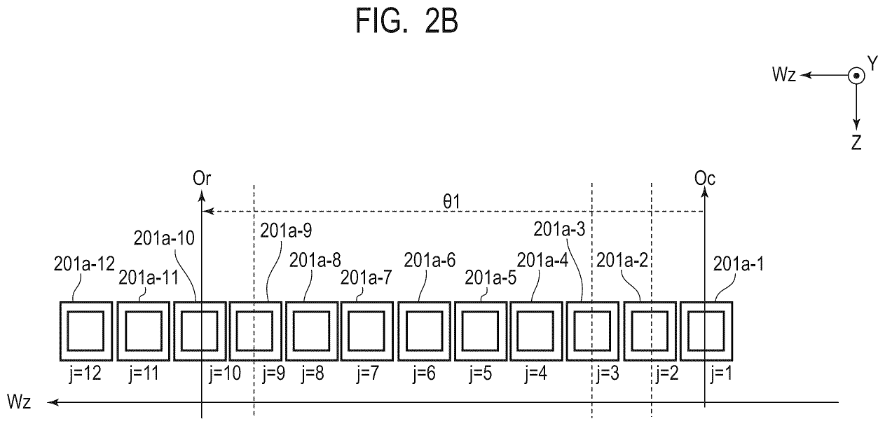

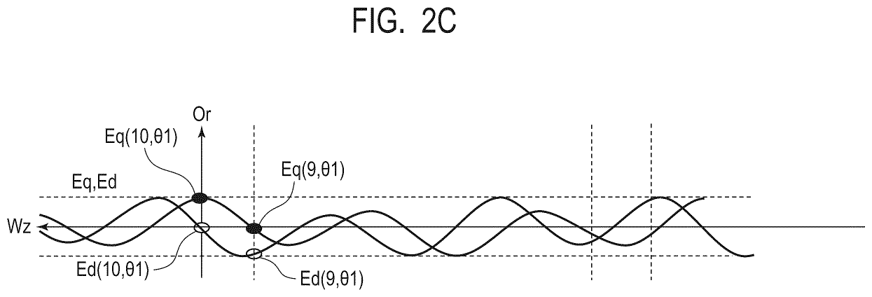

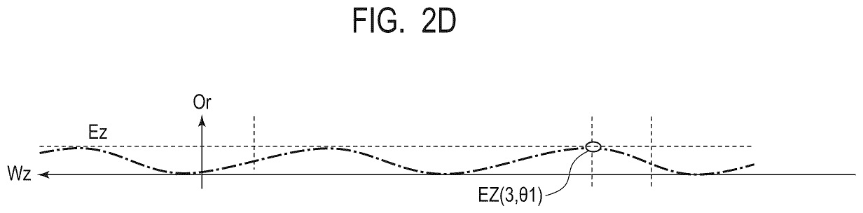

Here, using the first permanent magnets 101a and the first coils 201a as examples, the arrangement of these magnets and the thrust caused by them will be described with reference to FIG. 2A to FIG. 2D. Note that the arrangement and thrust of the second permanent magnets 101b and the second coils 201b are the same as those of the first permanent magnets 101a and the first coils 201a.

FIG. 2A is a transparent view of the arrangement of the plurality of first permanent magnets 101a viewed from the Y direction. In the present embodiment, as illustrated in FIG. 1B, twenty-four of first permanent magnets 101a are arranged. As illustrated in FIG. 2A, the twenty-four first permanent magnets 101a are arranged such that the first permanent magnets 101a-1 to the first permanent magnets 101a-24 are arranged at equal intervals in order to round the outer peripheral side surface of the rotor 110 in the Wz direction from the reference Or.

The first permanent magnets 101a are magnetized in the surface direction facing the first coils 201a. For example, the first permanent magnets 101a-1, 101a-3, 101a-19, and 101a-21 are magnetized to have the N-pole in the surface facing the first coil 201a, and the first permanent magnets 101a-18, 101a-22, and 101a-24 are magnetized to have the S-pole in the surface facing the first coil 201a. Further, for example, the first permanent magnets 101a-2, 101a-17, 101a-20, and 101a-23 are divided into two portions in the Z-direction, and are magnetized to have the N-pole and the S-pole in the respective portions.

Thus, the plurality of first permanent magnets 101a are arranged so that, on the side facing the first coils 201a, the magnetization units, in which the N-pole, the two-division magnetization, the N-pole, the S-pole, the two-division magnetization, and the S-pole are sequentially aligned in the Wz direction, are periodically aligned in the Wz direction.

Note that the plurality of first permanent magnets 101a may include a first permanent magnet line with different magnetic poles aligned alternately in the Wz direction, which is the rotation direction, and a second permanent magnet line with different magnetic poles aligned alternately in the Z direction, which is a direction intersecting the Wz direction. The Z direction is a direction along the Z-axis, which is the rotational axis. In FIG. 2A, the first permanent magnets 101a-1, 101a-3, 101a-18, 101a-19, 101a-21, 101a-22, and 101a-24 constitute the first permanent magnet line. In FIG. 2A, each of the first permanent magnets 101a-2, 101a-17, 101a-20, 101a-23 constitutes the second permanent magnet line. The second permanent magnet line is not limited to the permanent magnet of two-division magnetization as illustrated in FIG. 2A, but may be composed of a plurality of permanent magnets. In this case, the plurality of permanent magnets constituting the second permanent magnet line are arranged so that different magnetic poles are alternately arranged in the Z direction, which is a direction intersecting the Wz direction. In the second permanent magnet line, different magnetic poles may be alternately arranged not only in the Z direction but also in any direction intersecting with the Wz direction.

FIG. 2B is a transparent view of the arrangement of the first coil 201a viewed from the Y direction. In the present embodiment, as illustrated in FIG. 1B, twelve of the first coils 201a are arranged. As illustrated in FIG. 2B, the twelve first coils 201a are arranged so that the first coils 201a-1 to the first coils 201a-12 are sequentially aligned at equal intervals from the reference Oc in the Wz direction in an arc shape.

FIG. 2C and FIG. 2D are diagrams schematically illustrating the magnitudes of torques in a q-axis direction, a d-axis direction and the Z direction generated per unit current in the first coil 201a when the rotation angle θ of the rotor 110 is at θ1, that is, thrust constants Eq, Ed and Ez. The q-axis and the d-axis indicated here correspond to a q-axis and a d-axis in the motor control theory, respectively. FIG. 1B illustrates the directions of the q-axis and the d-axis with respect to the first coil 201a-10 as representative. The q-axis direction corresponds to the Wz direction, which is the circumferential direction, and the d-axis direction is the radial direction with the Z axis as the center. FIG. 2C illustrates the thrust constant Eq in the q-axis direction and the thrust constant Ed in the d-axis direction. FIG. 2D illustrates the thrust constant Ez in the Z direction.

As illustrated in FIG. 2C and FIG. 2D, the magnitude of each of the thrust constants Eq, Ed and Ez depends on the rotation angle θ1 of the rotor 110 and the index j of the first coil 201a. Here, the index j is a positive integer, and an integer satisfying 1≤ j≤12, for example. The first coil 201a can be identified by using the index j to denote “first coil 201a-j”. The thrust constants Eq, Ed, and Ez can be expressed as Eq (j, θ), Ed (j, θ), and Ez (j, θ), respectively, by displaying two arguments in parentheses, namely, the index j of the first coil 201a as the first argument and the rotation angle θ of the rotor 110 as the second argument. FIG. 2C and FIG. 2D illustrate the respective thrust constants when θ=θ1.

In the case illustrated in FIG. 2A and FIG. 2B where θ=θ1, the first coil 201a-10 is positioned substantially in the middle between the first permanent magnet 101a-24, whose facing face is S-pole, and the first permanent magnet 101a-1, whose facing face is N-pole. In this case, for example, a unit current is applied to the first coil 201a-10 so that the N-pole appears on the side facing the first permanent magnet 101a-24 and the first permanent magnet 101a-1. Note that the current is applied to the first coil 201a by a current control system 300 described later. When the unit current is applied to the first coil 201a-10 in this manner, an attractive force acts between the first coil 201a-10 and the first permanent magnet 101a-24, and a repulsive force acts between the first coil 201a-10 and the first permanent magnet 101a-1. That is, a thrust Eq (10, θ1) is applied to the first coil 201a-10 in the q-axis direction. On the other hand, a thrust Ed (10, θ1) applied to the first coil 201a-10 in the d-axis direction is relatively small.

Further, in the case illustrated in FIG. 2A and FIG. 2B, the first coil 201a-9 faces the first permanent magnet 101a-24, whose facing surface is an S pole. In this case, for example, a unit current is applied to the first coil 201a-9 so that the S-pole appears on the side facing the first permanent magnet 101a-24. When the unit current is applied to the first coil 201a-9 in this manner, a repulsive force is applied between the first coil 201a-9 and the first permanent magnet 101a-24. That is, a thrust Ed (9, θ1) is applied to the first coil 201a-9 in the d-axis direction. On the other hand, a thrust Eq (9, θ1) applied to the first coil 201a-9 in the q-axis direction is relatively small.

Further, in the cases illustrated in FIG. 2A and FIG. 2B, a unit current is applied to the first coil 201a-3 so that an N-pole appears on the side facing the first permanent magnet 101a-20. Then, since the first permanent magnet 101a-20 facing the first coil 201a-3 is magnetized by dividing it in the Z direction into the N-pole and the S-pole, a thrust Ez (3, θ1) in the Z-direction acts on the first coil 201a-3.

The force acting on the first coil 201a can be treated as equivalent to the reaction force acting on the rotor 110. Therefore, for example, when thrust is generated in the positive direction in the q-axis direction in the first coil 201a, thrust is generated in the negative direction in the q-axis direction in the rotor 110. Thus, the force acting on the rotor 110 can be controlled by the first coil 201a and the first permanent magnet 101a. In accordance with the rotation angle θ1, the forces in the q-axis direction, the d-axis direction and the Z-direction can be controlled by individually controlling the current applied to the coils using the respective thrust constants in the direction of control.

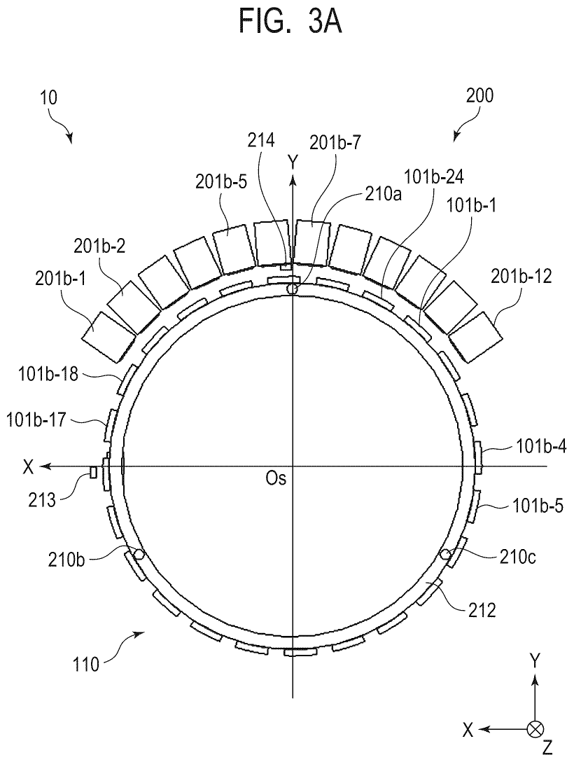

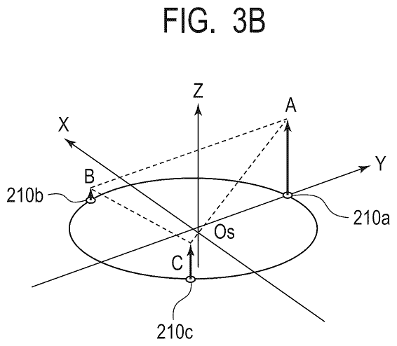

FIG. 3A is a side view of FIG. 1A viewed from the opposite side of FIG. 1B in the Z-direction. As illustrated in FIG. 3A, the Z-sensors 210a, 210b, and 210c are positioned laterally in the Z-direction with respect to the rotor 110 so as to face the circumferential end face of the rotor 110 in the Z-direction. The Z-sensors 210a, 210b, and 210c are arranged at equal angular intervals in the Wz direction, for example. The Z-sensors 210a, 210b, and 210c are arranged to detect the distance in the Z-direction to the rotor 110, respectively.

FIG. 3B is a diagram for explaining a method of calculating the displacements of the rotor 110 in the Z-direction, the Wx direction, and the Wy direction from the detection values of the Z-sensors 210a, 210b, and 210c. In calculating each displacement, a plane ABC including points A, B and C is formed based on the detected values of the Z sensors 210a, 210b, and 210c. The X and Y coordinates of the point A are the X and Y coordinates of the Z sensor 210a, respectively, and the Z coordinate of the point A is a coordinate based on the detected value of the Z sensor 210a. The X and Y coordinates of the point B are the X and Y coordinates of the Z sensor 210b, respectively, and the Z coordinate of the point B is a coordinate based on the detected value of the Z sensor 210b. The X and Y coordinates of the point C are the X and Y coordinates of the Z sensor 210c, respectively, and the Z coordinate of the point C is a coordinate based on the detected value of the Z sensor 210c. The displacement in the Z direction, the displacement in the Wx direction, and the displacement in the Wy direction of the plane ABC can be calculated from the inclination of the normal vector of the plane ABC and the distance from the origin Os to the plane ABC. The displacement of the plane ABC in the Z direction, the displacement in the Wx direction and the displacement in the Wy direction can be treated as the displacement of the rotor 110 in the Z direction, the displacement of the rotor 110 in the Wx direction and the displacement the rotor 110 in the Wy direction, that is, the position of the rotor 110 in the Z direction, the rotation angle the rotor 110 in the Wx direction and the rotation angle the rotor 110 in the Wy direction, respectively. Thus, the displacements of the rotor 110 can be calculated using the plane ABC. Note that respective displacements are calculated by a position controller 301 described below.

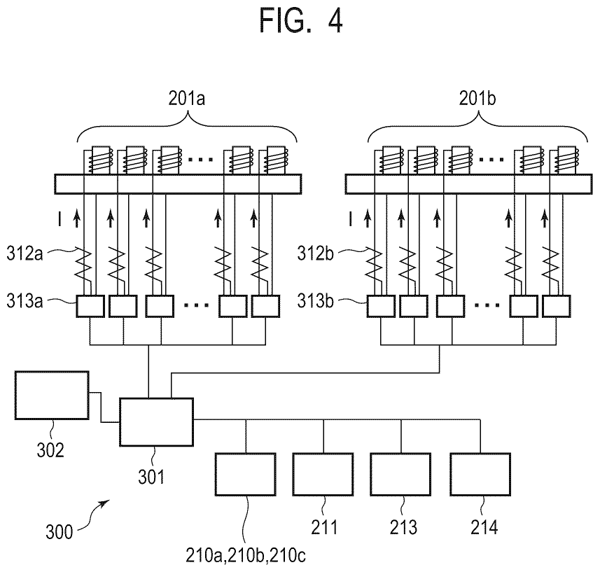

FIG. 4 is a connection diagram schematically illustrating a current control system 300 that controls a current applied to the first coils 201a and the second coils 201b. The current control system 300 will be described with reference to FIG. 4. The current control system 300 is a control unit that controls the drive of the rotor 110 by controlling the current applied to the plurality of first coils 201a and the plurality of second coils 201b. The configuration of the current control system 300 in connection with the first coils 201a will be described as an example, but the configuration in connection with the second coil 201b is also the same.

As illustrated in FIG. 4, the current control system 300 includes a levitation procedure executor 302, a position controller 301, a plurality of current sensors 312a, and a plurality of current controllers 313a. Current controllers 313b, current sensors 312b, and the second coils 201b are connected in the same way as the current controllers 313a, the current sensors 312a, and the second coils 201a.

The first coil 201a is individually connected to the current controller 313a. The current sensor 312a is individually connected to the current controller 313a. The current controller 313a can detect the current value of the first coil 201a by the current sensor 312a. Each of the current controllers 313a is connected to the position controller 301. The levitation procedure executor 302 generates and transmits a target position corresponding to the levitation procedure to the position controller 301. The levitation procedure will be described later.

The Z sensor 210a, 210b, and 210c, the Wz sensor 211, the X sensor 213, and the Y sensor 214 are connected to the position controller 301. The position controller 301 can detect the displacement X, Y, Z, Wx, Wy, and Wz of the rotor 110 based on the detection values of the respective sensors outputted from the Z sensors 210a, 210b, and 210c, the Wz sensor 211, the X sensor 213, and the Y sensor 214. The displacement X is a displacement in the X direction, the displacement Y is a displacement in the Y direction, the displacement Z is a displacement in the Z direction, the displacement Wx is a displacement in the Wx direction, the displacement Wy is a displacement in the Wy direction, and the displacement Wz is a displacement in the Wz direction. The position controller 301 detects the displacement X based on the detection value of the X sensor 213, the displacement Y based on the detection value of the Y sensor 214, and the displacement Wz based on the detection value of the Wz sensor 211. The position controller 301 detects the displacements Z, Wx, and Wy as described above based on the detection values of the Z sensors 210a, 210b, and 210c.

The position controller 301 includes a control program, clock, and the like inside it, and calculates the current value to be applied to each first coil 201a according to each displacement of the rotor 110. The position controller 301 transmits a current command value to each current controller 313a, which commands the calculated current value to be applied to each first coil 201a.

The current controller 313a individually applies a predetermined current to the corresponding first coil 201a while detecting the current amount by the current sensor 312a according to the current command value from the position controller 301.

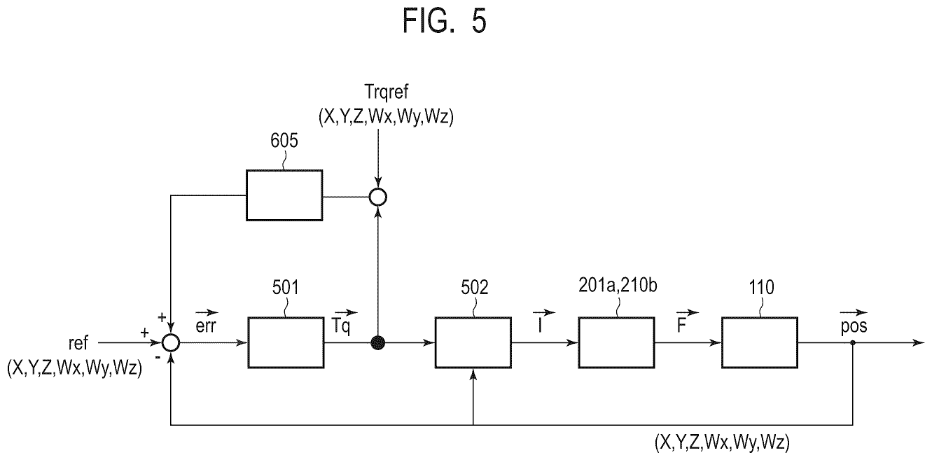

FIG. 5 schematically illustrates a control loop for calculating the magnitude of the torque applied to the rotor 110. A method of controlling the attitude of the rotor 110 by the position controller 301 will be described with reference to FIG. 5. In FIG. 5, “ref” is a target value of the displacement of the rotor 110, and “Trqref” is a target value of the torque applied to the rotor 110. Also, “pos” is the displacement (X, Y, Z, Wx, Wy, Wz) of the rotor 110 acquired from the detection value of the sensor group (the Z sensor 210a, 210b, and 210c, the Wz sensor 211, the X sensor 213, and the Y sensor 214). The position controller 301 includes an attitude controller 501, a current calculator 502, and a torque controller 605.

The torque controller 605 calculates an operation amount “d” to the target value from the difference between the torque “Tq” applied to the rotor 110 and the target value “Trqref” of the torque applied to the rotor 110. When the respective components in the Y direction, the Wx direction, and the Wz direction of the target values “Trqref” of Z, Wz, and Wy are 0 [N], the control works as so-called zero power control. The torque controller 605 performs so-called zero power control, and can set a position and an attitude in which the gravity and the attractive force applied to the rotor 110 are balanced as a target value.

The attitude controller 501 calculates a torque “Tq” applied to the rotor 110 from the difference “err” between the target value “ref” and the operation amount “d”, and the displacement “pos”. The attitude controller 501 may be, for example, a controller by PID control or a controller using a filter as appropriate according to the characteristics of the rotor 110. Such a controller can stabilize the attitude of the rotor 110.

The current calculator 502 determines the current “I” applied to the first coil 201a and the second coil 201b from the torque “Tq” and the displacement “pos”. The current controllers 313a and 313b apply the determined current “I” to the first coil 201a and the second coil 201b, respectively.

When a current “I” is applied to the first coil 201a and the second coil 201b, an electromagnetic force “F” is generated between the first coil 201a and the second coil 201b and the rotor 110 to act on the rotor 110. Thus, levitation control and rotation control of the rotor 110 are performed. The attitude controller 501 detects displacement “pos” again from the detection values of the Z sensors 210a, 210b, and 210c, the Wz sensor 211, the X sensor 213, and the Y sensor 214, and repeats the above processing. In this way, the levitation control and the rotation control of the rotor 110 are performed to control the drive of the rotor 110.

The torque vector “Tq” indicating the torque applied to the rotor 110 as described above is expressed by the following Expression (1). Tx, Ty, and Tz are the magnitudes of the torque in the X direction, the torque in the Y direction, and the torque in the Z direction, respectively. Twx, Twy, and Twz are the magnitudes of the moments of force around the X-axis, the Y-axis, and the Z-axis, respectively. The torque here includes the force and the moment of force, and the torque along an axis such as in the X direction, the Y direction, the Z direction, or the like is the force.

Tq=(Tx,Ty,Tz,Twx,Twy,Twz) Expression (1)

The rotation drive apparatus 10 according to the present embodiment controls the current flowing through the first coils 201a and the second coils 201b by the current control system 300 to control the respective components Tx, Ty, Tz, Twx, Twy, and Twz of the torque vector “Tq”. Thus, the rotation drive apparatus 10 can rotate the rotor 110 in the Wz direction while controlling the attitude (X, Y, Z, Wx, Wy) of the rotor 110. Note that, in the attitude of the rotor 110, X, Y and Z are positions in the X direction, the Y direction and the Z direction, respectively, and Wx and Wy are rotation angles around the X-axis and the Y-axis, respectively.

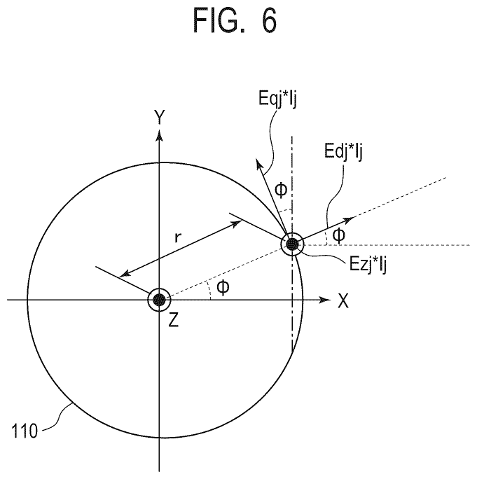

FIG. 6 is a diagram for explaining torques generated in the q-axis direction, the d-axis direction and the Z direction at an arbitrary position of the rotor 110. Here, FIG. 6 is used to explain that the torque vector “Tq” can be applied to the rotor 110 at an arbitrary rotation angle in the Wz direction of the rotor 110 by moving a magnetic field generated in the first coils 201a and the second coils 201b in accordance with the rotation of the rotor 110.

In the present embodiment, since the first coils 201a and the second coils 201b are arranged in the stator 200, torques can be generated in the q-axis direction, the d-axis direction and the Z direction. As illustrated in FIG. 2A, each of the plurality of first permanent magnets 101a and the plurality of second permanent magnets 101b constitute a permanent magnet line including permanent magnets divided in the Z direction and permanent magnets not divided in the Z direction, respectively. In the permanent magnet line, the permanent magnets not divided in the Z direction mainly contribute to the torque in the q-axis direction and the d-axis direction, and the permanent magnets divided in the Z direction mainly contribute to the torque in the Z direction.

Here, symbols used for the explanation are summarized. In FIG. 6, the symbols are shown as appropriate.
j: index for distinguishing the coils in the first coils 201a or the second coils 201b (j=1 to 12)
Ij: current value applied to the j-th first coil 201a-j or the j-th second coil 201b-j
Φj: angle in the Wz direction of the j-th first coil 201a-j or the j-th second coil 201b-j
r: radius to the first permanent magnet 101a or the second permanent magnet 101b
*: multiplication symbol
Σ: sum when the index j is changed from 1 to 12

Symbols for forces acting on the first coil 201a are as follows. Eqj, Edj and Ezj shown in FIG. 6 correspond to Eqa (j, θ), Eda (j, θ) and Eza (j, θ) shown below, respectively.
Eqa (j, θ): force per unit current in the q-axis direction acting between the j-th first coil 201a-j and the rotor 110 at the rotation angle θ
Eda (j, θ): force per unit current in the d-axis direction acting between the j-th first coil 201a-j and the rotor 110 at the rotation angle θ
Eza (j, θ): force per unit current in the Z direction acting between the j-th first coil 201a-j and the rotor 110 at rotation angle θ

Symbols for forces acting on the second coil 201b are as follows. Eqj, Edj and Ezj shown in FIG. 6 correspond to Eqb(j, θ), Edb(j, θ) and Ezb(j, θ) shown below, respectively.
Eqb(j, θ): force per unit current in the q-axis direction acting between the j-th second coil 201b-j and the rotor 110 at the rotation angle θ
Edb(j, θ): force per unit current in the d-axis direction acting between the j-th second coil 201b-j and the rotor 110 at the rotation angle θ
Ezb(j, θ): force per unit current in the Z direction acting between the j-th second coil 201b-j and the rotor 110 at the rotation angle θ

The torque vector Tq generated in the rotor 110 is represented by Expression (1) shown above. The components of the torque vector generated by the first coils 201a are represented by Txa, Tya, Tza, and Twza, and the components of the torque vector generated by the second coils 201b are represented by Txb, Tyb, Tzb, and Twzb. These components are components in the following directions.
Txa: component in the X direction of the torque vector generated by the first coils 201a
Tya: component in the Y direction of the torque vector generated by the first coils 201a
Tza: component in the Z direction of the torque vector generated by the first coils 201a
Twza: component in the Wz direction of the torque vector generated by the first coils 201a
Txb: component in the X direction of the torque vector generated by the second coils 201b
Tyb: component in the Y direction of the torque vector generated by the second coils 201b
Tzb: component in the Z direction of the torque vector generated by the second coils 201b
Twzb: component in the Wz direction of the torque vector generated by the second coils 201b

The components Txa, Tya, Tza, and Twza are represented by the following Expressions (2-1) to (2-4), respectively.

Txa=Σ{(−Eqa(j,θ)*sinΦj+Eda(j,θ)*cosΦj)*Ij} Expression (2-1)

Tya=Σ{(Eqa(j,θ)*cosΦj+Eda(j,θ)*sinΦj)*Ij} Expression (2-2)

Tza=Σ{Eza(j,θ)*Ij} Expression (2-3)

Twza=Σ{Eqa(j,θ)*r*Ij} Expression (2-4)

The components Txb, Tyb, Tzb, and Twzb are represented by the following Expressions (2-5) to (2-8), respectively.

Txb=Σ{(−Eqb(j,θ)*sinΦj+Edb(j,θ)*cosΦj)*Ij} Expression (2-5)

Tyb=Σ{(Eqb(j,θ)*cosΦj+Edb(j,θ)*sinΦj)*Ij} Expression (2-6)

Tzb=Σ{Ezb(j,θ)*Ij} Expression (2-7)

Twzb=Σ{Eqb(j,θ)*r*Ij} Expression (2-8)

Here, as illustrated in FIG. 1C, it is assumed that the first coils 201a and the second coils 201b are arranged at a distance t from the XY-plane in the Z direction in plane symmetry with the XY-plane as the plane of symmetry. Then, the components Tx, Ty, Tz, Twx, Twy, and Twz of the torque vector Tq are represented by the following Expressions (3-1) to (3-6), respectively.

Tx=Txa+Txb Expression (3-1)

Ty=Tya+Tyb Expression (3-2)

Tz=Tza+Tzb Expression (3-3)

Twx=(Tya−Tyb)*t Expression (3-4)

Twy=(Txa−Txb)*t Expression (3-5)

Twz=Twza+Twzb Expression (3-6)

To apply the desired torque vector Tq, the current Ij satisfying the above Expressions (2-1) to (2-8) and (3-1) to (3-6) may be applied to each of the first coils 201a and the second coils 201b. The current control system 300 can control the application of the current Ij as such. Note that each coil generates some torque in the d-axis direction. The torque in the d-axis direction generated by each coil can be cancelled out by arranging the first coils 201a and the second coils 201b in plane symmetry with the XY-plane and the YZ-plane as the planes of symmetrical, because the directions of the torques are opposite to each other.

In addition to the control described above, the currents applied to the coils can be further controlled by the current control system 300 to further apply the force of Twy to the rotor 110. Specifically, the plurality of first coils 201a are arranged on the upstream side and the downstream side with respect to the YZ-plane, and the currents of the first coils 201a facing the first permanent magnets 101a divided in the Z direction can be controlled so as to generate opposite torques on the upstream side and the downstream side.

For example, in FIG. 1C, FIG. 2A, and FIG. 2B, the first coils 201a-1 to 201a-6 are arranged on the upstream side and the first coils 201a-7 to 201a-12 are arranged on the downstream side. The first permanent magnets 101a-20 and 101a-23 divided in the Z direction are positioned so as to face the first coils 201a-3 and 201a-8, respectively. Note that the relationship between the second coils 201b and the second permanent magnets 101b is the same. In this case, in order to obtain the torque in the Z direction, the currents are applied to the first coils 201a facing the first permanent magnets 101a divided in the Z direction. When both the first coil 201a-3 on the upstream side and the first coil 201a-8 on the downstream side are applied with the currents in the same direction, the torque in the Z direction can be obtained as shown in Expression (3-3) above. On the other hand, for example, when the first coil 201a-3 on the upstream side is applied with the current in the positive direction, and the first coil 201a-8 on the downstream side is applied with the current in the negative direction, the torques generated in the Z direction are opposite on the upstream side and the downstream side, and thus the torque in the Wy direction can be obtained. Note that, since the first coil 201a-12 also faces the first permanent magnet 101a-2 divided in the Z direction, the torque may be obtained by adding the first permanent magnet 101a-2 in the same manner.

FIG. 2A and FIG. 2B illustrate a case where the first permanent magnets 101a-2, 101a-20, and 101a-23 divided in the Z direction, which is the second permanent magnet line, face the first coils 201a-1 to 201a-12. When the rotor 110 rotates in the Wz direction, the first permanent magnets 101a also rotate in the Wz direction. The torque Tz in the Z direction is controlled by the currents flowing through the first coils 201a facing the first permanent magnets 101a divided in the Z direction. Therefore, when the rotor 110 rotates in the Wz direction, it is desirable that the plurality of first permanent magnets 101a are arranged so that at least one or more of the first permanent magnets 101a divided in at least the Z direction face the plurality of first coils 201a arranged in an arc shape. The relative positional relationship between the first coils 201a and the first permanent magnets 101a makes it possible to always obtain the torque Tz in the Z direction. That is, when the first permanent magnets 101a divided in the Z direction face one or more of the first coils 201a-1 to 201a-12, the torque Tz in the Z direction can always be obtained regardless of the rotation angle θ of the rotor 110. Thus, the torque Tz in the Z direction can always be obtained during rotation of the rotor 110, so that stable rotation control of the rotor 110 can be performed. Note that the relative positional relationship between the second coils 201b and the second permanent magnets 101b can be made similar to the relative positional relationship between the first coils 201a and the first permanent magnets 101a described above.

Here, the distances from the YZ-plane to the points where the first permanent magnets 101a are divided in the Z direction on the upstream side and the downstream side are h1 and h2, respectively. Note that the distances h1 and h2 change with the rotation when the rotor 110 is rotating.

The torque Tza (j=1 to 6) in the Z direction generated by the first coil 201a-1 to 201a-6 on the upstream side and the torque Tzb (j=1 to 6) in the Z direction generated by the second coil 201b-1 to 201b-6 on the upstream side are obtained by the following Expressions (4-1) and (4-2), respectively. In the Expressions (4-1) and (4-2), ¿ is the sum when the index j is changed from 1 to 6.

Tza(j=1 to 6)=Σ{Eza(j,θ)*Ij} Expression (4-1)

Tzb(j=1 to 6)=Σ{Ezb(j,θ)*Ij} Expression (4-2)

The torque Tza (j=7 to 12) in the Z direction generated by the first coils 201a-7 to 201a-12 on the downstream side is obtained by the following Expression (4-3). The torque Tzb (j=7 to 12) in the Z direction generated by the second coil 201b-7 to 201b-12 on the downstream side is obtained by the following Expression (4-4). In the Expressions (4-3) and (4-4), Σ is the sum when the index j is changed from 7 to 12.

Tza(j=7 to 12)=Σ{Eza(j,θ)*Ij} Expression (4-3)

Tzb(j=7 to 12)=Σ{Ezb(j,θ)*Ij} Expression (4-4)

Then, the torque Twy in the Wy direction represented by the following Expression (4-5) is obtained by Expression (3-5) and Expressions (4-1) to (4-4).

Twy=(Txa−Txb)*t+{(Tza(j=1 to 6)+Tzb(j=1 to 6))*h1−(Tza(j=7 to 12)+Tzb(j=7 to 12))*h2} Expression (4-5)

Thus, the rotation drive apparatus 10 according to the present embodiment can control the rotational speed and attitude of the rotor 110 because the current control system 300 can apply the torque vector Tq having the components of six axes to the rotor 110 as described above.

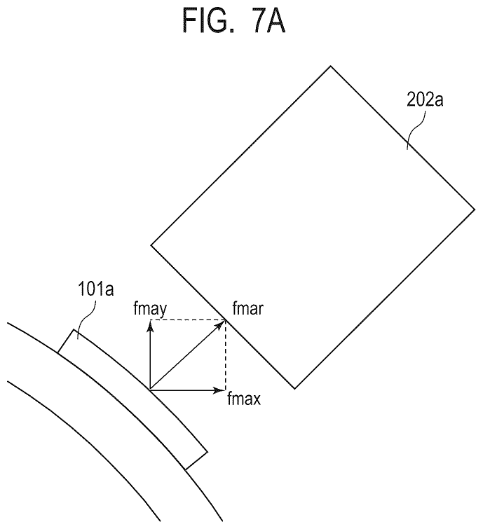

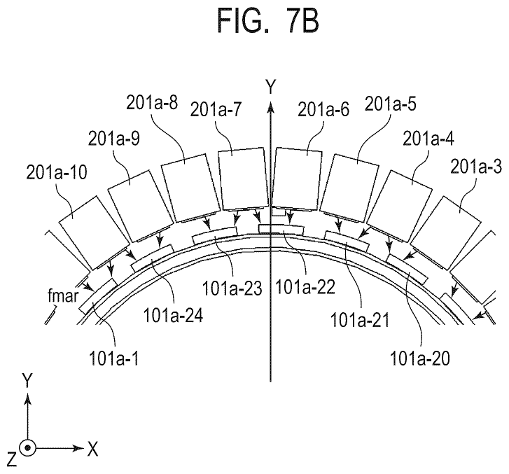

FIG. 7A and FIG. 7B are schematic diagrams illustrating the attractive force acting between the first permanent magnet 101a of the rotor 110 and the core 202a of the first coil 201a. The attractive force acting between the core 202a of the first coil 201a and the first permanent magnet 101a will be described with reference to FIG. 7A and FIG. 7B. Note that the second coil 201b and the second permanent magnet 101b have the same structure, and the same attractive force acts between them.

The core 202a arranged within the first coil 201a is made of a magnetic material. Therefore, as illustrated in FIG. 7A, an attractive force “fmar”, which is a magnetic force, is generated between the core 202a and the first permanent magnet 101a. The attractive force “fmar” is decomposed into an attractive force “fmax” in the X direction and an attractive force “fmay” in the Y direction. The magnitude and direction of the attractive force “fmar” depend on the relative positional relationship between the core 202a and the first permanent magnet 101a, as illustrated by arrows in FIG. 7B.

According to the configuration of the present embodiment, in the Y direction which is the direction of gravity, the combined force obtained by adding the attractive force “fmay” acting on the first coils 201a-1 to 201a-12 in the Y direction becomes the attractive force “Fmay” generated in the direction of gravity of the rotor 110.

On the other hand, in the X direction, since the first coils 201a-1 to 201a-6 and the first coils 201a-7 to 201a-12 are arranged in plane symmetry with the YZ-plane as the plane of symmetry, attractive force is also generated in plane symmetry with the YZ-plane as the plane of symmetry. Specifically, in the X direction, the attractive forces act in the positive direction in the first coils 201a-1 to 201a-6 and in the negative direction in the first coils 201a-7 to 201a-12. When the rotor 110 is centered in the X direction, the attractive forces in the positive direction and the attractive forces in the negative direction in the X direction cancel each other. That is, in this case, the combined force of the attractive forces “fmax” in the X direction acting on the first coils 201a-1 to 201a-6 and the combined force of the attractive forces “fmax” in the X direction acting on the first coils 201a-7 to 201a-12 have approximately the same magnitudes to cancel out each other.

In the present embodiment, as described above, the first coils 201a have the cores 202a, which causes the attractive force “Fmay” to act on the rotor 110 in the Y direction. Also, the second coils 201b have the cores 202b, which causes the attractive force “Fmby” to act on the rotor 110 in the Y direction as well as the attractive force “Fmay. The attractive forces “Fmay” and “Fmby” in the Y direction are used as a force for levitating the rotor 110 in the Y direction. Note that the first coil 201a and the second coil 201b are not necessarily limited to a cored coil having a core, and may be an air-core coil. In the case of the air core coil, the torque Ty in the Y direction is used as a force for levitating the rotor 110 in the Y direction.

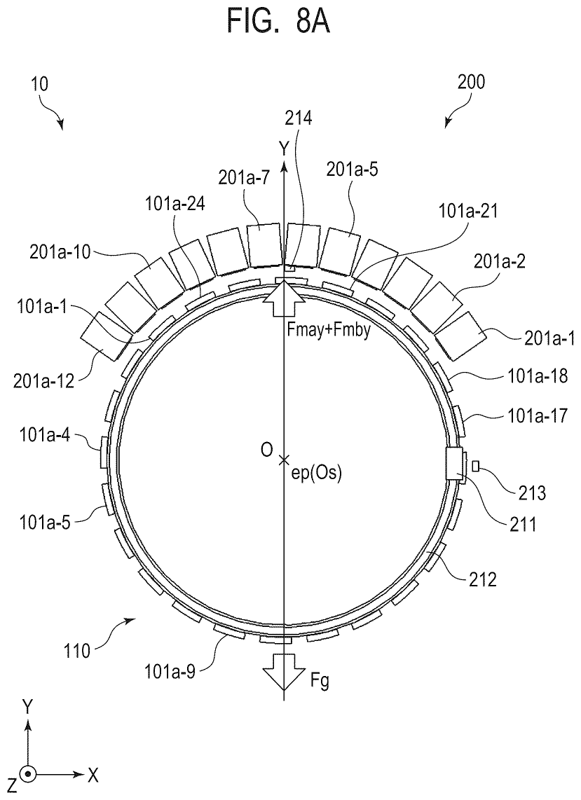

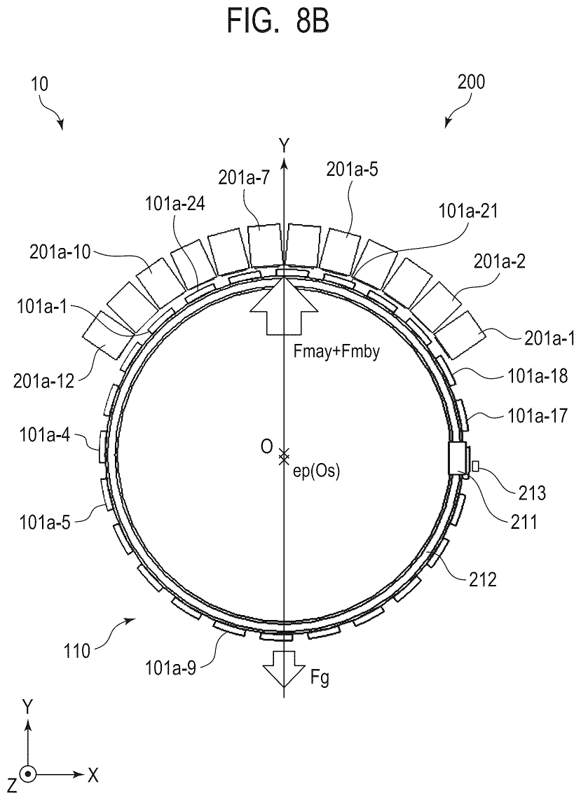

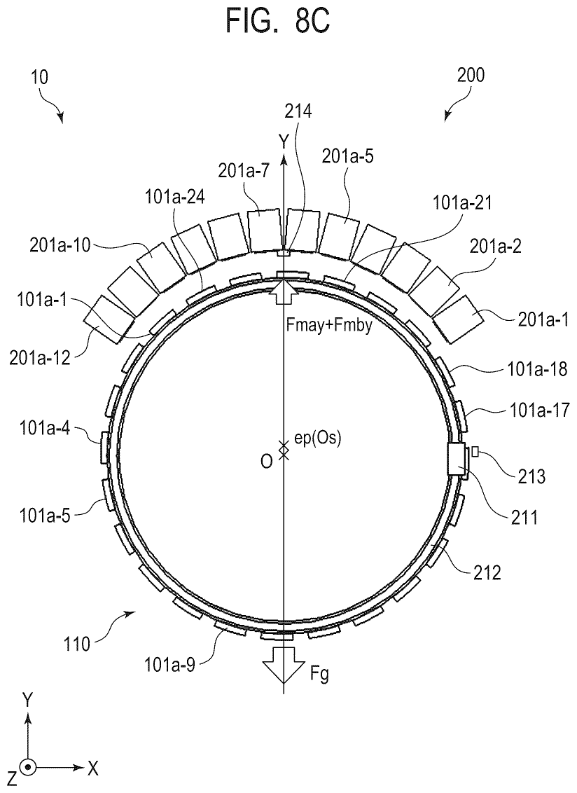

FIG. 8A, FIG. 8B, and FIG. 8C are schematic diagrams of the movement of the rotor 110 in the Y direction with the center position of the rotor 110 in the X direction aligned with the origin Os. The levitation of the rotor 110 will be described with reference to FIG. 8A to FIG. 8C.

The rotor 110 is arranged at a position where its center O overlaps with the origin Os. When the rotor 110 is moved in the Y direction, there is an equilibrium point “ep” at which the magnitude of the gravity Fg of the rotor 110 is equal to the magnitude of the attractive forces “Fmay”+ “Fmby”. Note that, in the present embodiment, the origin Os and the equilibrium point “ep” are configured to overlap with each other. FIG. 8A illustrates a state in which the rotor 110 is arranged so that the center O of the rotor 110 overlaps with the equilibrium point “ep”.

For example, when the position of the rotor 110 is shifted in the positive direction in the Y direction as illustrated in FIG. 8B from the state illustrated in FIG. 8A, the attractive force “Fmay”+ “Fmby” becomes larger than the gravity force “Fg”. On the other hand, the position of the rotor 110 is shifted in the negative direction in the Y direction as illustrated in FIG. 8C from the state illustrated in FIG. 8A, the gravity force “Fg” becomes larger than the attractive force “Fmay”+ “Fmby”. The rotor 110 moves in the direction of the larger force 110, that is, in the direction of the attractive force “Fmay” + “Fmby” in the case illustrated in FIG. 8B, and in the direction of the gravity force “Fg” in the case illustrated in FIG. 8C.

Therefore, the rotation drive apparatus 10 applies currents to the first coils 201a and the second coils 201b by the current control system 300 at a position where the center O of the rotor 110 overlaps with the equilibrium point “ep” to control the torque in the Y direction applied to the rotor 110. Further, the rotation drive apparatus 10 also controls the torque applied to the rotor 110 in the X direction in the same manner to maintain the levitation of the rotor 110.

In order to maintain the levitation of the rotor 110, it is desirable to control the torque applied to the rotor 110 at the position where the center O of the rotor 110 overlaps the equilibrium point “ep”. When the control is performed at the equilibrium point “ep” in this way, the levitation of the rotor 110 can be continued at smaller currents as the currents flowing through the first coils 201a and the second coils 201b. Further, by applying currents to the first coils 201a and the second coils 201b so as to generate the torque in the Wz direction as described above, the rotor 110 can rotate in the Wz direction while keeping the rotor 110 levitated so that the center O of the rotor 110 overlaps the equilibrium point “ep”. Thus, the rotation drive apparatus 10 can rotate the rotor 110 in the Wz direction while levitating the rotor 110 in the Y direction by controlling the current flowing through the first coils 201a and the second coils 201b by the current control system 300 to control the coils with respect to the rotor 110.

To arrange the rotor 110 at the position where the center O of the rotor 110 overlaps the equilibrium point “ep”, the torque Ty in the Y direction can be controlled by applying currents to the first coils 201a and the second coils 201b. In this case, when the rotor 100 is not controlled, seating bearings 220 for preventing the rotor 110 from falling are required.

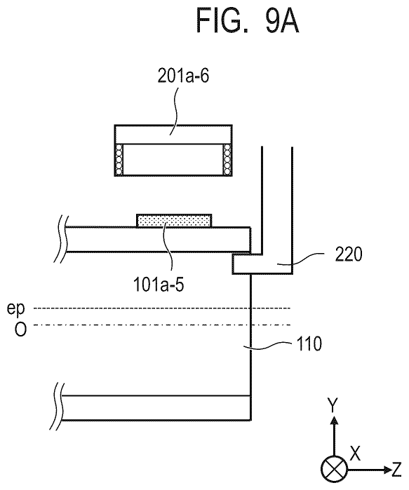

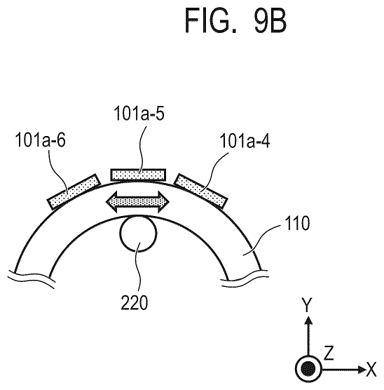

The seating bearings 220 will be described with reference to FIG. 9A and FIG. 9B together with a state in which the rotor 110 comes into contact with the seating bearings 220. FIG. 9A is a sectional view illustrating a cross section in which the rotor 110 is cut in the YZ-plane including the center axis including the center O of the rotor 110, and illustrates a state in which the rotor 110 comes into contact with the seating bearing 220. FIG. 9B is a side view of the state illustrated in FIG. 9A viewed from the Z direction.

As illustrated in FIG. 9A and FIG. 9B, the rotation drive apparatus 10 according to the present embodiment further includes seating bearings 220. The seating bearings 220 are installed so as to function as supports that supports the rotor 110 when the rotor 100 is not controlled. Specifically, the seating bearings 220 are installed on one end side and the other end side of the rotor 100 in the Z direction. When the rotor 100 is not controlled, the seating bearings 220 support the upper side walls of one end and the other end of the cylindrical rotor 100 in the Z direction from the hollow interior side of the rotor 100 to prevent the rotor 110 from falling.

In the case illustrated in FIG. 9A and FIG. 9B, the rotor 110 is in a position lower than the equilibrium point “ep”, and the rotor 110 comes into contact with the seating bearings 220 by gravity. The seating bearing 220 supports the rotor 110 coming into contact therewith from below to prevent the rotor 110 from falling. In this case, the rotor 110 comes into contact with the seating bearings 220 but is not fixed thereto. Therefore, it is possible that the rotor 110 moves while sliding in the X direction. In such a case, when the torque Ty is applied to the rotor 110 and a force other than the direction of gravity is simultaneously applied to the rotor 110, the rotor 110 also moves in the X direction. As a result, when the rotor 110 comes into contact with the seating bearing 220 again due to the movement in the X direction, vibration occurs in the rotor 110, and the transition from the contact state of the rotor 110 to the levitation state may fail.

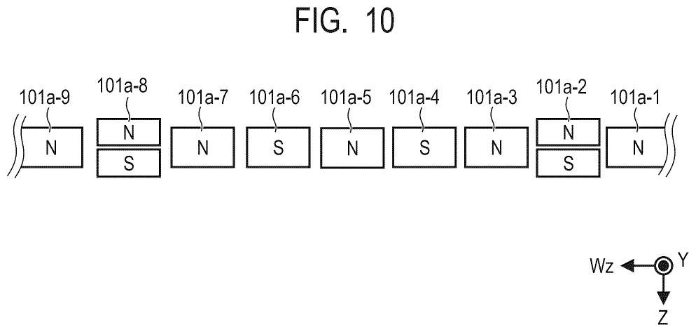

The difference between a state in which the transition of the rotor 110 to the levitation state can be stably performed and a state in which the transition of the rotor 110 to the levitation state is unstable will be described with reference to FIG. 10 to FIG. 14. In the following description, the first permanent magnets 101a and the first coils 201a will be taken as examples, but the same is also true for the second permanent magnets 102b and the second coils 201b.

FIG. 10 illustrates, as an example of the arrangement of the first permanent magnets 101a, a case where the first permanent magnets 101a for control in the Z direction are arranged every six magnets in the magnet line.

In this case, the first permanent magnets 101a-2 and 101a-8 are for control in the Z direction. When currents are applied to the first coils 201a facing the first permanent magnets 101a-2 and 101a-8, the rotor 110 moves in the Z direction. Therefore, when the rotor 110 is shifted from the contact state to the levitation state, the torque Ty must be applied to the rotor 110 by using the permanent magnets 101a other than the first permanent magnets 101a-2 and 101a-8.

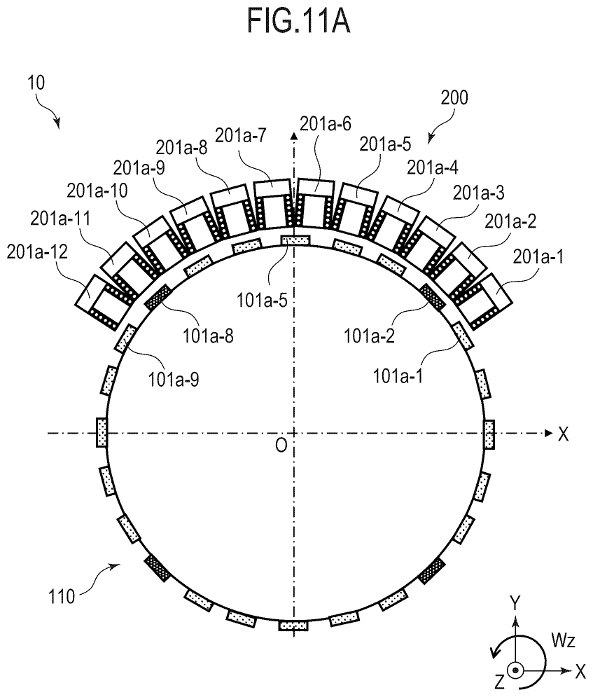

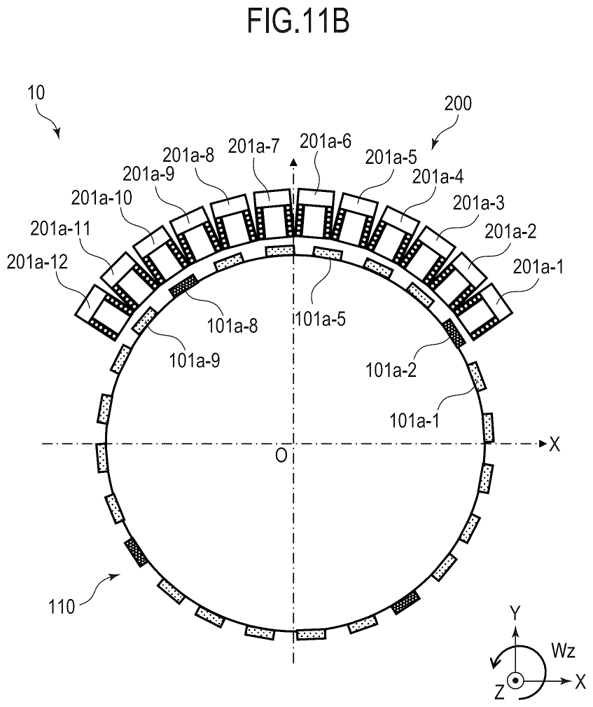

FIG. 11A and FIG. 11B are side views of the rotation drive apparatus 10 viewed in the Z direction, respectively, and illustrate two patterns of examples of the electric angle of the rotor 110. As will be described below, the electric angle of the rotor 110 illustrated in FIG. 11A is an angle in which the arrangement of the first permanent magnets 101a has a predetermined symmetry with respect to the Y-axis. The electric angle of the rotor 110 illustrated in FIG. 11B is an angle in which the arrangement of the first permanent magnets 101a has a predetermined asymmetry with respect to the Y-axis. The electric angle of the rotor 110 corresponds to the position of the rotor 110 in the Wz direction, which is the rotation direction.

As illustrated in FIG. 11A, the first permanent magnets 101a-2 and 101a-8 for control in the Z direction are arranged in line symmetry with respect to the Y-axis in a plan view viewed in the Z direction. The first permanent magnet 101a-5 is arranged on the Y-axis. Therefore, the magnetic pole direction of the first permanent magnet 101a-5 is parallel to the direction of gravity. The first permanent magnets 101a-1, 101a-3 to 101a-7, and 101a-9, other than the first permanent magnets 101a-2 and 101a-8, are also arranged in line symmetry with respect to the Y-axis in the plan view viewed in the Z direction. The first coils 201a-1 to 201-12, which face the first permanent magnets 101a-1 to 101a-9, are also arranged in line symmetry with respect to the Y-axis in the plan view viewed in the Z direction. Thus, the first permanent magnets 101a-1 to 101a-9 and the first coils 201a-1 to 201-12 are arranged in line symmetry with respect to the Y-axis in the plan view viewed in the Z direction.

In the state of the electric angle illustrated in FIG. 11A, the first coils 201a-1, 201a-3 to 201a-10, and 201a-12 are coils that can generate the torque Ty without generating the torque Tz. These first coils 201a-1, 201a-3 to 201a-10, and 201a-12 that can generate the torque Ty are arranged in line symmetry with respect to the Y-axis in the plan view viewed in the Z direction. Therefore, the torque Tx generated by the first coils 201a-1, 201a-3 to 201a-10, and 201a-12 can be easily cancelled out among the plurality of coils. As a result, in the case illustrated in FIG. 11A, the torque Ty can be efficiently obtained without disturbance.

On the other hand, in the case illustrated in FIG. 11B, the first permanent magnets 101a-2 and 101a-8 for controlling in the Z direction are arranged asymmetrically with respect to the Y-axis in a plan view viewed in the Z direction. In this case, unlike the case illustrated in FIG. 11A, the first coils 201a-2 to 201a-9, 201a-11, and 201a-12 are coils that can generate the torque Ty without generating the torque Tz. These first coils 201a-2 to 201a-9, 201a-11, and 201a-12 are arranged asymmetrically with respect to the Y-axis in the plan view viewed in the Z direction. Therefore, when equal currents are applied to all of the first coils 201a, the torque Tx in the X direction is generated, and therefore, it is necessary to balance the torque Tx by weakening the applied currents to some of the plurality of first coils 201a. As a result, in the state illustrated in FIG. 11B, as compared with the state illustrated in FIG. 11A, the state of the apparatus tends to become unstable when the rotor 110 changes from the state in contact with the seating bearings 220 to the levitation state due to the disturbance caused by the generation of the torque Tx and the lowering of the torque Ty caused by the weakening of the currents.

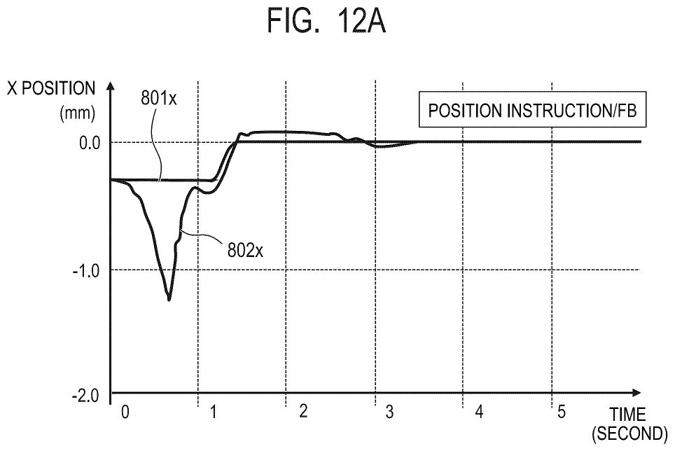

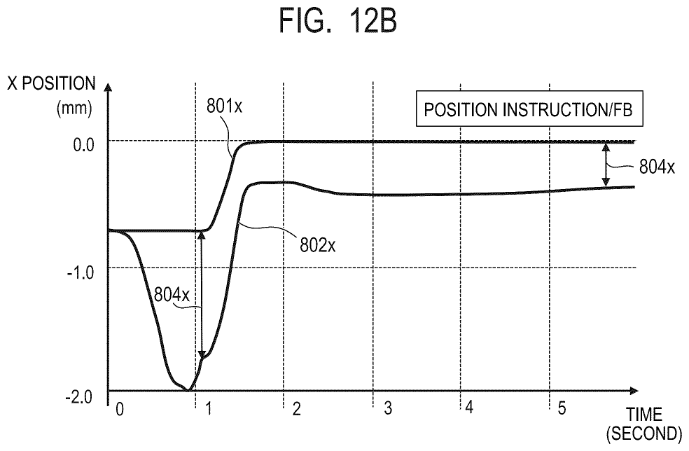

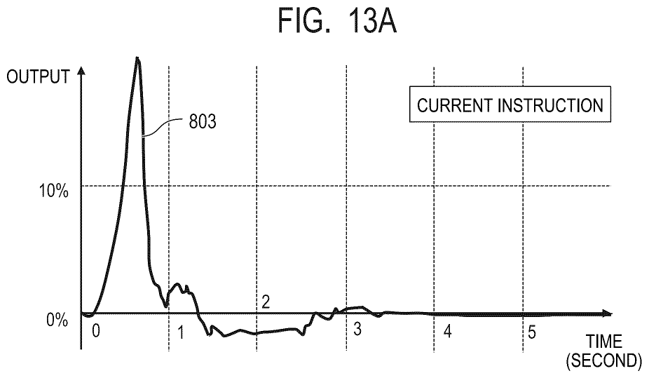

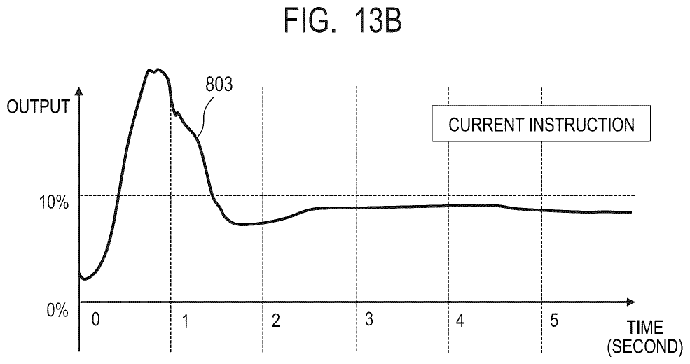

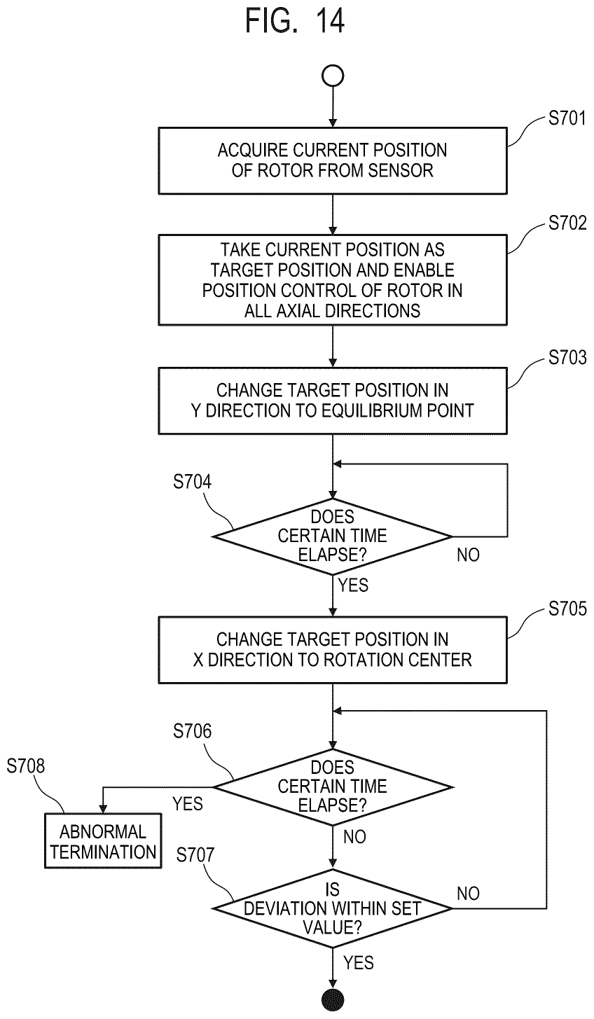

The stability of the levitation of the rotor 110 when levitated from the state of the electric angle illustrated in FIG. 11A and the stability of the levitation of the rotor 110 when levitated from the state of the electric angle illustrated in FIG. 11B will be compared with each other and described with reference to FIG. 12A to FIG. 14. FIG. 12A and FIG. 12B are graphs that show examples of data of the position control of the rotor 110 illustrated in FIG. 11A and FIG. 11B, respectively. FIG. 13A and FIG. 13B are graphs that show examples of date of current control in FIG. 12A and FIG. 12B, respectively. FIG. 14 is a flowchart illustrating the levitation procedure by the levitation procedure executor 302 illustrated in FIG. 4.

In FIG. 12A and FIG. 12B, the vertical axis indicates position of the rotor 110 in the X direction, and the horizontal axis indicates the time axis with time 0 seconds as the time when the position control of the rotor 110 in the Y direction was started. FIG. 12A and FIG. 12B show the position command value 801x and the X position 802x. The position command value 801x is a position command value in the X direction generated by the position controller 301 illustrated in FIG. 4. The X position 802x indicates the X position of the rotor 110 detected by the X sensor 213 illustrated in FIG. 4. The X position 802x is used as feedback to the position controller 301 (indicated as “FB” in the figure). Hereinafter, the position detected by the sensor is referred to as the FB position. By performing control in accordance with the procedure illustrated in FIG. 14, the position command value 801x and the FB position 802x transition over time as illustrated in FIG. 12A and FIG. 12B. With the change in the position command value 801x shown in FIG. 12A and FIG. 12B, the current command value 803 by the position controller 301 transitions over time as shown in FIG. 13A and FIG. 13B, respectively.

A levitation procedure for levitating the rotor 110 will be described with FIG. 14 together with reference to FIG. 12A and FIG. 12B. FIG. 14 is a flowchart illustrating the levitation procedure of the rotor 110. The levitation procedure illustrated in FIG. 14 is executed by the levitation procedure executor 302.

As illustrated in FIG. 14, the levitation procedure executor 302 acquires the current position of the rotor 110 from the Z sensor 210, the Wz sensor 211, the X sensor 213, and the Y sensor 214 via the position controller 301 (Step S701).

Next, the levitation procedure executor 302 takes the acquired current position as the target position and enables the position control of the rotor 110 in all axial directions (Step S702). All axial directions include the X direction, the Y direction, the Z direction, the Wx direction, the Wy direction, and the Wz direction. Here, when the rotor 110 comes into contact with the seating bearings 220, the position of the rotor 110 is stable because the rotor 110 is supported by the seating bearings 220 in addition to the position control.

Next, in order to shift the rotor 110 from the seating state to the levitation state, the target position in the Y direction in the position control is changed to the equilibrium point (Step S703). In FIG. 12A and FIG. 12B, this time point is the time 0 seconds. When the rotor 110 starts to be levitated in accordance with the change of the target position in the Y direction, the position of the rotor 110 in the X direction is unintentionally fluctuated because the support by the seating bearings 220 is lost. Since this position fluctuation in the X direction can be restored by the position control, the levitation procedure executor 302 waits until a certain time elapses (Step S704). In the examples of FIG. 12A and FIG. 12B, the time 0 seconds to 1 second is the interval of Step S704.

In the interval of Step S704, there is a difference in the behavior of the rotor 110 between the case illustrated in FIG. 12A and the case illustrated in FIG. 12B.

First, in the case illustrated in FIG. 12A, since the arrangement of the first permanent magnets 101 illustrated in FIG. 11A is linearly symmetrical with respect to the Y-axis in the plan view viewed in the Z direction, the torque Tx in the X direction due to the application of the torque Ty in the Y direction is not generated easily, and the control of the rotor 110 in the X direction is easy. Therefore, the FB position 802x is once deviated from the position command value 801x, but after 1 second has elapsed, the FB position 802x can be returned to the vicinity of the position command value 801x by the position control.

On the other hand, in the case illustrated in FIG. 12B, the first permanent magnets 101 illustrated in FIG. 11B is asymmetrical with respect to the Y-axis in the plan view viewed in the Z direction, so that the torque Tx in the X direction occurs during the application of the torque Ty in the Y direction. Therefore, the FB position 802x cannot return to the vicinity of the position command value 801x, resulting in a steady-state deviation 804x, which is a steady difference between the FB position 802x and the position command value 801x.

Subsequently, as illustrated in FIG. 14, the levitation procedure executor 302 changes the target position in the X direction of the position control to the origin Os as the rotation center of the rotor 110 (Step S705). Since the target position in the X direction before Step S705 was the position before the start of the position control read by the sensor, the position depended on the use state of the rotor 110 immediately before and was not reproducible. On the other hand, since the target position is changed to the rotation center in step S705 and thereafter, the target position is the same even when the operation is repeated. Therefore, in the cases illustrated in FIG. 12A and FIG. 12B, the position command values 801x in the X direction at the time 0 seconds do not coincide with each other, but at time 2 seconds and later, the operations coincide with each other.

Here, focusing on the FB position 802x in the X direction, the FB position 802x can follow the change of the position command value 801x in the case illustrated in FIG. 12A. On the other hand, in the case illustrated in FIG. 12B, a deviation continues to occur between the position command value 801x and the FB position 802x even after the change of the position command value 801x, resulting in the steady-state deviation 804x.

After changing the target position in the X direction to the rotation center, the levitation procedure executor 302 repeats the determination whether or not a predetermined certain time has elapsed (Step S706) and the determination whether or not the deviation between the position command value 801x and the FB position 802x is within a predetermined set value (Step S707).

If the levitation procedure executor 302 determines that the certain time has not elapsed (Step S706, NO) and determines that the deviation is within the set value (Step S707, YES), the levitation procedure executor 302 determines that the rotor 110 has moved to the rotation center and has succeeded in the transition from the seated state to the levitation state.

On the other hand, if the levitation procedure executor 302 determines that the deviation is not within the set value (Step S707, NO) and determines that the certain time has elapsed (Step S706, YES), the levitation procedure executor 302 determines that the levitation has failed and abnormally terminates the control of the rotor 110 (Step S708). In the case illustrated in FIG. 12B, the steady-state deviation of 804x occurs. Therefore, in the case illustrated in FIG. 12B, there is a high possibility that it is necessary to configure the levitation procedure executor 302 to determine that the rotor 110 cannot rotate due to eccentricity depending on the magnitude of the steady-state deviation of 804x, and to determine an abnormal end.

The influence of the steady-state deviation of 804x also appears in the current command value 803 calculated by the current calculator 502.

FIG. 13A illustrates the transition of the current command value 803 in the same time axis as FIG. 12A. In this case, the current command value 803 temporarily rises in accordance with the movement of the rotor 110 in the X direction in FIG. 12A at the time of the transition from the seated state to the levitation state, but after the position of the rotor 110 in the X direction reaches the target position, the current command value 803 remains at almost zero. This indicates that after the transition to the levitation state, the rotor 110 can be levitated stably at the center of rotation even if almost no current is applied to the coils 201.

On the other hand, FIG. 13B illustrates the transition of the current command value 803 in the same time axis as FIG. 12B. In this case, the current command value 803 does not return to zero, which indicates that the current must continue to flow through the coils 201 in order for the rotor 110 to remain near the rotation center. In addition, although the current continues to flow through the coil 201, the steady-state deviation 804x remains in FIG. 12B. The current that constantly flows through the coils 201 to produce the torque Tx uses a portion of the current capacity of the coils 201. Therefore, the ratio of the current that produces the torque Ty to the total current decreases. Here, the ratio of the current that can contribute to the torque Ty to the current capacity of the coils 201 is defined as the levitation force. The levitation force with respect to the rotor 110 will be used for the evaluation of the levitating stability of the rotor 110. Note that the levitation force may be defined as a force in the Y direction acting on the rotor 110 corresponding to the ratio of the current that can contribute to the torque Ty to the current capacity of the coil 201. The levitation force is based on the electric angle of the rotor 110, and specifically changes with the electric angle of the rotor 110.

In the position controller 301, it is possible to reduce the steady-state deviation 804x by increasing the current value flowing through the coils 201 by increasing the gain during control. However, in this would result in problems such as an increase in power consumption and a risk of oscillation due to the increased gain. Therefore, the gain of the position controller 301 cannot be easily increased. A method of levitating the rotor 110 from the seating bearing 220 at a position where the steady-state deviation 804x does not occur is required.

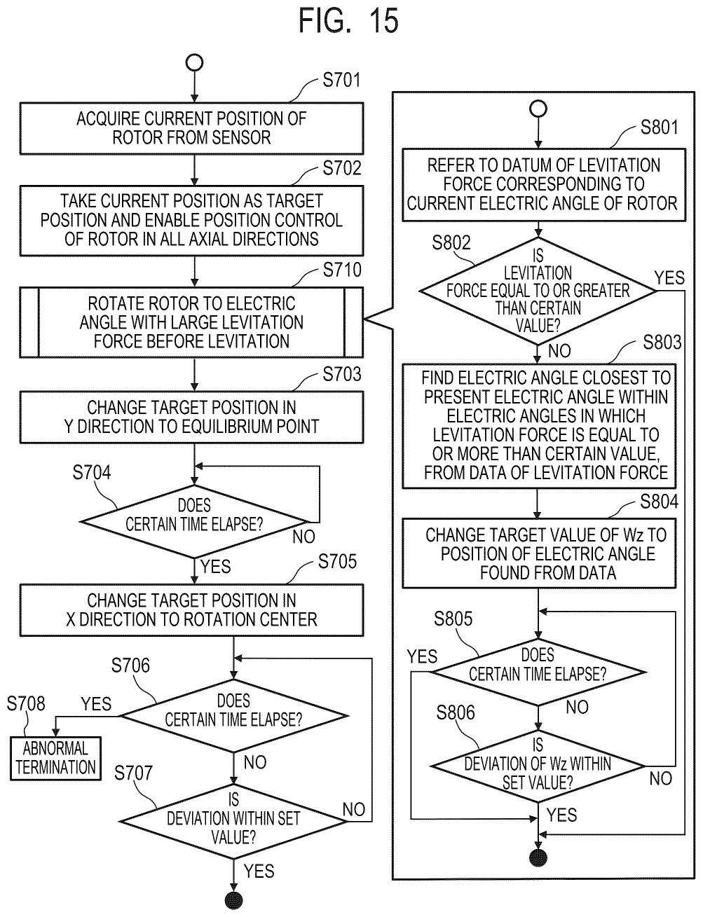

Therefore, in the present embodiment, a procedure for rotating the rotor 110 in the Wz direction while keeping the rotor 110 in contact with the seating bearings 220 is added to the levitation procedure illustrated in FIG. 14 to prevent the steady-state deviation 804x from occurring. FIG. 15 illustrates a flowchart in which the procedure for rotating the rotor 110 in the Wz direction is added. The levitation procedure illustrated in FIG. 15 is executed by the levitation procedure executor 302.

In addition to the procedures from Step S701 to Step S707 illustrated in FIG. 14, the levitation procedure illustrated in FIG. 15 includes a procedure for rotating the rotor 110 before levitation (Step S71θ) between the procedure of Step S702 and the procedure of Step S703. The procedure of Step S710 includes the procedures from Step S801 to Step S806. Details of the procedure of Step S710 will be described in the order of Step S801 to Step S806.

The electric angle of the rotor 110 is determined from the current position of the rotor 110 acquired in Step S701. Therefore, the levitation procedure executor 302 determines the electric angle of the rotor 110 from the current position of the rotor 110 acquired in Step S701, and refers to the datum of the levitation force corresponding to the electric angle (Step S801). Here, the data of the levitation force is a table of levitation force against the rotor 110 at each electric angle measured in advance at the stage of the adjustment of the rotation drive apparatus 10. The levitation procedure executor 302 can refer to the data of the levitation force against the rotor 110 stored in a storage device (not illustrated). Note that the data of the levitation force is not limited to those acquired by measurement, but may be calculated based on the design value of the magnetic circuit determined from the positional relationship between the stator 200 and the rotor 110.

Next, the levitation procedure executor 302 determines whether the datum of the levitation force acquired by reference is equal to or greater than a predetermined certain value (Step S802).

If the levitation procedure executor 302 determines that the datum of the levitation force is equal to or greater than the certain value (Step S802, YES), the levitation procedure executor 302 ends the procedure of Step S710 in the middle. As illustrated in FIG. 11A, when the permanent magnets 101 are arranged in line symmetry with respect to the Y-axis in the plan view viewed in the Z direction, the steady-state deviation in the X direction when the rotor 110 is levitated is negligibly small. Therefore, in such a case, most of the current capacity of the coil 201 can be allocated to the torque Ty, and the levitation force becomes a certain value or more. Therefore, the levitation force is sufficiently secured, and the rotor 110 can be levitated stably at the current electric angle. Therefore, in the case illustrated in FIG. 11A where the datum of the levitation force is determined to be the certain value or more, the levitation procedure executor 302 ends the procedure of Step S710 in the middle.

If the levitation procedure executor 302 determines that the datum of the levitation force is less than the certain value (Step S802, NO), the levitation procedure executor 302 finds an electric angle in which levitation force is equal to or more than the certain value and which is closest to the present electric angle from the data of the levitation force (Step S803). The electric angle found here is for rotating the rotor 110 before levitation.

As a candidate of the electric angle found in Step S803, that is, a candidate of the target position in the Wz direction, there is an electric angle in which the permanent magnets 101 are arranged in line symmetry with respect to the Y-axis in the plane view viewed in the Z direction. There are two possible candidates of such an electric angle as follows. That is, as illustrated in FIG. 11A, there are two possible electric angles in which the first permanent magnets 101a-2 and 101a-8 for controlling in the Z direction are positioned in line symmetry with respect to the Y-axis in the plan view viewed in the Z direction, and the electric angle in which the first permanent magnet 101a-8 for controlling in the Z direction overlaps with the Y-axis. However, in the latter case, since the two first coils 201a-6 and 201a-7, which can most efficiently transmit the torque Ty to the rotor 110, face the first permanent magnet 101a-8 for controlling in the Z direction, levitation force is reduced as compared with the former case. Therefore, the electric angle illustrated in FIG. 11A is found and selected as the electric angle having a high levitation force.

Next, the levitation procedure executor 302 changes the target position of the position controller 301 in the Wz direction to the value of the electric angle found in the procedure of Step S803 (Step S804). When the target position in the Wz direction is changed, the position controller 301 starts the rotation of the rotor 110 in the Wz direction. That is, the position controller 301 starts the rotation of the rotor 110 in the Wz direction while the rotor 110 remains in contact with the seating bearings 220 in the case of the arrangement of the permanent magnet 101 asymmetrical to the Y-axis in the plan view viewed in the Z direction as illustrated in FIG. 11B. At this time, the position controller 301 may apply the torque Ty in the Y direction to the rotor 110 such that the rotor 110 is not levitated for the purpose of reducing friction between the rotor 110 and the seating bearings 220.

After changing the target position in the Wz direction, the levitation procedure executor 302 repeats a determination whether or not a predetermined certain time has elapsed (Step S805) and a determination whether or not the deviation between the position command value and the FB position in the Wz direction is within a predetermined set value (Step S806).

If the levitation procedure executor 302 determines that the certain time has not elapsed (Step S805, NO) and determines that the deviation in the Wz direction is within the set value (Step S806, YES), the levitation procedure executor 302 confirms the completion of the movement to the electric angle set as the target position and ends the procedure of Step S710. Thus, the rotation of the rotor 110 to the electric angle where the levitation force is equal to or more than the certain value is completed.

On the other hand, if the levitation procedure executor 302 determines that the deviation in the Wz direction is not within the set value (Step S806, NO) and determines that the certain time has elapsed (Step S805, YES), the levitation procedure executor 302 ends the procedure of Step S710. In this case, the deviation in the Wz direction does not fall within the set value within the certain time, and the timeout determination is made. However, it is highly likely that the timeout determination is made due to friction between the rotor 110 and the seating bearings 220, and the possibility remains that the rotor 110 can be successfully levitated even when the deviation in the Wz direction is not within the set value. Therefore, even when the timeout determination is made, the levitation procedure executor 302 ends the procedure of Step S710 without determining that it is abnormal.

After the completion of the procedure of Step S710, the levitation procedure executor 302 executes the procedures of Step S703 to Step S707 as in the case illustrated in FIG. 14.

As described above, in the present embodiment, in the procedure of Step S710 of FIG. 15, the levitation force at the electric angle of the rotor 110 to be controlled is checked before the levitation starts. As a result, when it is determined that the levitation force is insufficient, the rotor 110 is rotated in advance while being seated to change the electric angle. This prevents the torque Tx in the X direction from being applied to the rotor 110 at the start of levitation to cause a steady-state deviation and oscillation, while the torque Ty in the Y direction can be efficiently transmitted to the rotor 110. Thus, according to the present embodiment, the rotor 110 can be transitioned from the seated state to the levitation state stably while avoiding the enlargement of the whole rotation drive apparatus 10.

Note that, in the procedure illustrated in FIG. 15, the rotor 110 is rotated before the levitation, but at the end of the levitation, the rotor 110 may be rotated and stopped in advance for the next levitation. Specifically, when the rotor 110 is shifted from a levitation state to a seated state in contact with the seating bearings 220, the levitation procedure executor 302 may rotate the rotor 110 to an electric angle where the levitation force becomes a certain value or more when the rotor 110 is supported by the seating bearings 220. After the rotation, the levitation procedure executor 302 can make the rotor 110 supported by the seating bearing 220 to stop the rotor 110 and end the control of the rotor 110. In this case, since it is not necessary to rotate the rotor 110 at the start of levitation, the waiting time when the rotation drive apparatus 10 is used can be shortened.

### Second Embodiment

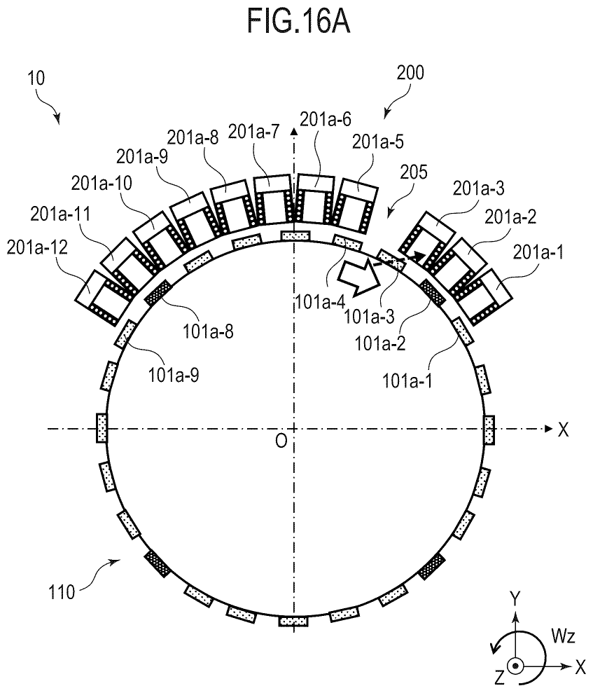

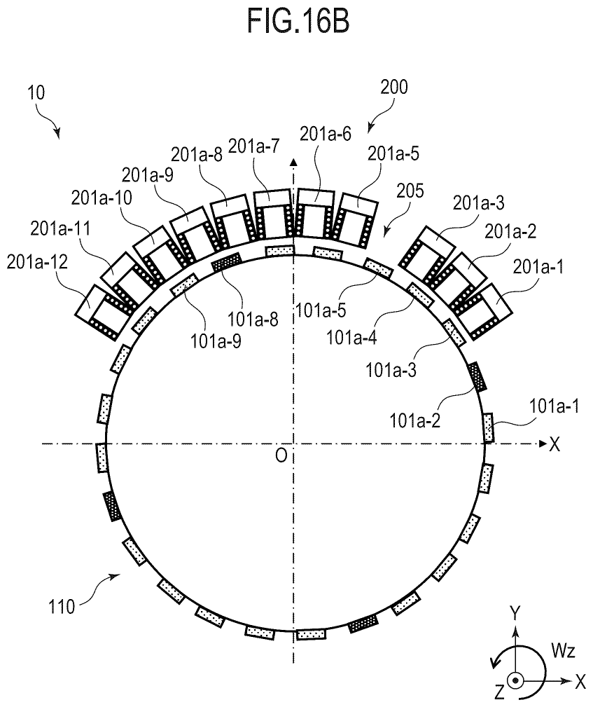

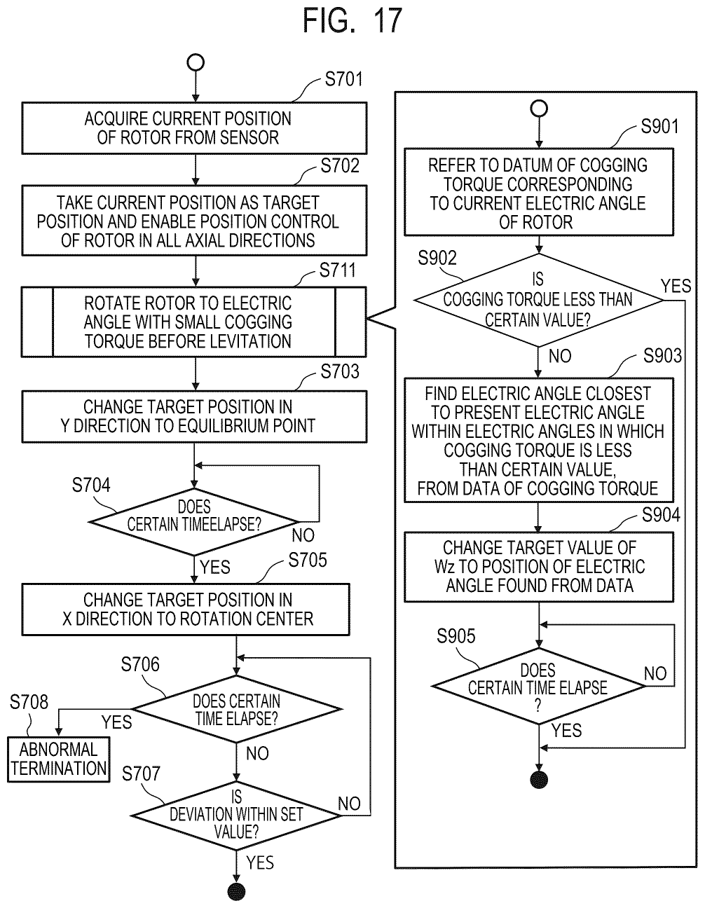

A rotation drive apparatus according to a second embodiment of the present disclosure will be described with reference to FIG. 16A to FIG. 17. Note that the same components as those in the above first embodiment are labeled with the same references, and the description thereof will be omitted or simplified.

The basic configuration of the rotation drive apparatus 10 and the basic control method of the rotor 110 according to the present embodiment are the same as those according to the first embodiment. The arrangement of the first permanent magnets 101 is the same as that illustrated in FIG. 10 according to the first embodiment. In the present embodiment, a case where the coils 201 as the magnetic force units are not arranged at equal intervals will be described.

When the magnetic force units such as the coils or the like are arranged on the stator, it is ideal to arrange the magnetic force units at equal intervals in order to reduce cogging torque. In practice, however, there are cases where the magnetic force units cannot be arranged at equal intervals due to the restriction of the arrangement of equipment in the apparatus.

FIG. 16A illustrates an example where the coils 201 cannot be arranged at equal intervals in the configuration illustrated in FIG. 11A. Comparing FIG. 11A and FIG. 16A, the structures of the rotor 110 are the same as each other. On the other hand, with regard to the structure of the stator 200, in FIG. 16A, the first coil 201a-4 is not arranged due to interference with equipment in the apparatus, resulting in a void 205. Thus, in the plurality of first coils 201a illustrated in FIG. 16A, the void 205 is formed between the first coils 201a adjacent to each other. In this respect, the arrangement of the first coils 201a illustrated in FIG. 16A is different from the arrangement illustrated in FIG. 11A and the arrangement of the first coils 201a illustrated in FIG. 16A is asymmetrical with respect to the Y-axis in the plan view viewed from the Z direction.

The space where the coil 201 is arranged has low magnetoresistance because the core of the coil 201 exists. On the other hand, magnetoresistance is high in the void 205. Therefore, attraction is generated between the first permanent magnet 101a-3 and the core in the first coil 201a-3, and torque in the −Wz direction is generated in the rotor 110 without applying current to the first coil 201a. Note that, when the rotor 110 is in contact with the seating bearings 220, friction is generated between the rotor 110 and the seating bearing 220, so that the rotor 110 remains stationary even when the torque in the −Wz direction is generated.

When attempting to levitate the rotor 110 from the state illustrated in FIG. 16A in accordance with the flowchart illustrated in FIG. 14, after the procedure of Step S703 for changing the target position in the Y direction to the equilibrium point is executed, the rotor 110 is levitated from the seating bearings 220. Therefore, friction between the rotor 110 and the seating bearings 220 is eliminated. Then, the torque in the −Wz direction suddenly affects the rotor 110 at the moment of levitation, so that the rotor 110 vibrates greatly. A method of levitating the rotor 110 stably from the seating bearings 220 is required while preventing the rotor 110 from vibrating at the moment of levitation.

Therefore, in the present embodiment, a procedure for rotating the rotor in the Wz direction while contacting the seating bearing 220 is added to the levitation procedure illustrated in FIG. 14 to prevent cogging torque to the rotor 110 from occurring. The cogging torque to the rotor 110 includes torque in the X direction and the Wz direction. The cogging torque is based on the electric angle of the rotor 110, and is specifically changed by the electric angle of the rotor 110. FIG. 17 illustrates a flowchart in which a procedure for rotating the rotor 110 in the Wz direction is added. The levitation procedure illustrated in FIG. 17 is executed by the levitation procedure executor 302.

In addition to the procedures from Steps S701 to Step S707 illustrated in FIG. 14, the levitation procedure illustrated in FIG. 17 includes a procedure for rotating the rotor 110 before levitation (Step S711) between the procedure of Step S702 and the procedure of Step S703. The procedure of Step S711 includes the procedures from Step S901 to Step S905. Details of the procedure of Step S711 will be described in the order of Step S901 to Step S905.

The electric angle of the rotor 110 is determined from the current position of the rotor 110 acquired in Step S701. Therefore, the levitation procedure executor 302 determines the electric angle of the rotor 110 from the current position of the rotor 110 acquired in Step S701, and refers to the datum of cogging torque corresponding to the electric angle (Step S901). Here, the cogging torque data for the rotor 110 is a table of cogging torque measured in advance at each electric angle at the stage of the adjustment of the rotation drive apparatus 10. Note that the data of the cogging torque is not limited to those acquired by measurement, but may be calculated based on the design value of the magnetic circuit determined from the positional relationship between the stator 200 and the rotor 110.

Next, the levitation procedure executor 302 determines whether the datum of the cogging torque acquired by reference is less than a predetermined certain value (Step S902).

If the levitation procedure executor 302 determines that the datum of the cogging torque is less than the certain value (Step S902, YES), the levitation procedure executor 302 ends the procedure of Step S711 in the middle. In the example of FIG. 16B, the plurality of first permanent magnets 101a are arranged so that one of the first permanent magnets 101a does not face each other across both of the first coils 201a-3 or 201a-5 adjacent to the void 205 and the void 205. In this way, when there is no permanent magnet 101 partially facing the void 205 and the coil 201, the cogging torque can be reduced to the same level as when there is no void 205 in the coils 201. In this case, the value of the cogging torque is less than a certain value. Therefore, the rotor 110 can be levitated stably at the current electric angle. Therefore, when the cogging torque data is determined to be less than the certain value as illustrated in FIG. 16B, the levitation procedure executor 302 ends the procedure of Step S711 in the middle.

On the other hand, if the levitation procedure executor 302 determines that the datum of the cogging torque is equal to or more than the certain value (Step S902, NO), the levitation procedure executor 302 finds an electric angle in which the cogging torque is less than the certain value and which is closest to the current electric angle from the data of the cogging torque (Step S903). The electric angle found here is for rotating the rotor 110 before levitation. As illustrated in FIG. 16A, when the first permanent magnet 101a-3 partially faces the first coil 201a-3 near the void 205 in the first coils 201a, the cogging torque has a large value. In such a case, the levitation procedure executor 302 determines that the datum of the cogging torque is equal to or more than the certain value.

As a candidate of the electric angle found in Step S903, that is, a candidate of the target position in the Wz direction, there is an electric angle in which the first permanent magnets 101a are arranged so as not to make the first permanent magnet 101a partially face the first coil 201a, as illustrated in FIG. 16B. Further, even if such an arrangement does not exist in the design of the apparatus, the cogging torque is reduced in the electric angle in which the torque in the Wz direction generated between each permanent magnet 101 and the coil 201 is cancelled out in the positive direction and the negative direction opposite each other. Therefore, the electric angle in which the torque in the Wz direction generated between the plurality of permanent magnets 101 and the plurality of coils 202 is cancelled out is also a candidate of the target position.

Next, the levitation procedure executor 302 changes the target position of the position controller 301 in the Wz direction to the value of the electric angle found in the procedure of Step S903 (Step S904). When the target position in the Wz direction is changed, the position controller 301 starts the rotation of the rotor 110 in the Wz direction. That is, the position controller 301 starts the rotation of the rotor 110 in the Wz direction while the rotor 110 remains in contact with the seating bearings 220. At this time, the position controller 301 may apply the torque Ty in the Y direction such that the rotor 110 is not levitated for the purpose of reducing friction between the rotor 110 and the seating bearings 220.

After changing the target position in the Wz direction, the levitation procedure executor 302 repeats a determination (Step S905) as to whether or not a predetermined certain time has elapsed. If the levitation procedure executor 302 determines that the certain time has elapsed (Step S905, YES), the levitation procedure executor 302 confirms the completion of the movement to the electric angle set as the target position and ends the procedure of Step S711. Thus, the rotation of the rotor 110 to the electric angle where the cogging torque becomes less than the certain value is completed.

After the completion of the procedure of Step S711, the levitation procedure executor 302 executes the procedures of Step S703 to Step S707 as in the case illustrated in FIG. 14.

As described above, in the present embodiment, in the procedure of Step S711 of FIG. 17, the cogging torque at the electric angle of the rotor 110 to be controlled is checked before the levitation starts. As a result, when it is determined that the cogging torque greatly affects the realization of stable levitation, the rotor 110 is rotated in advance while being seated to change the electric angle. This prevents the torque in the X direction or the Wz direction due to cogging to the rotor 110 from being applied to the rotor 110 at the start of levitation to prevent oscillation in the rotor 110. Thus, according to the present embodiment, the rotor 110 can be transitioned from the seated state to the levitation state stably while avoiding the enlargement of the whole rotation drive apparatus 10.

Note that the levitation procedure of the rotor 110 illustrated in FIG. 17 can be executed not only when the coils 201 have the void 205 but also when the coils 201 as exemplified in FIG. 11A of the first embodiment does not have any void 205.

In addition to the examples illustrated in FIG. 16A and FIG. 16B, the arrangement of the coils 201 in the stator 200 may be asymmetric with respect to the Y-axis in a plan view viewed in the Z direction, and the positions of the coils 201 may be slightly shifted when assembling the apparatus even if the arrangement is linearly symmetric in design. In these cases, the torque Tx or the torque Ty must be continuously applied to the rotor 110 in order to keep the rotor 110 levitating at the origin Os, which is the rotation center. In these cases, it is desirable to keep the rotor 110 levitating at the magnetic equilibrium point rather than at the coordinate center. Therefore, switching the control of the rotor 110 from the position control to the zero power control after the transition from the seated state to the levitation state is also effective for stabilizing the levitating of the rotor 110.

Also note that, in the procedure illustrated in FIG. 17, the rotor 110 is rotated before the levitation, but at the end of the levitation, the rotor 110 may be rotated and stopped in advance for the next levitation. Specifically, when the rotor 110 is shifted from a levitation state to a seating state in contact with the seating bearings 220, the levitation procedure executor 302 may rotate the rotor 110 to an electric angle where the cogging torque is less than the certain value when the rotor 110 is supported by the seating bearings 220. After the rotation, the levitation procedure executor 302 can make the rotor 110 supported by the seating bearings 220 to stop the rotor 110 and end the control of the rotor 110. In this case, since it is not necessary to rotate the rotor 110 at the start of levitation, the waiting time when the rotation drive apparatus 10 is used can be shortened.

### Third Embodiment

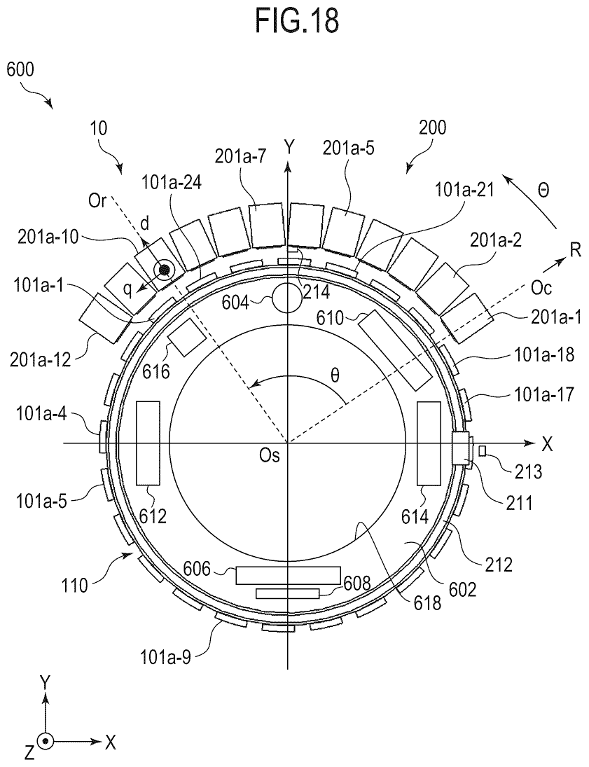

Rotation equipment according to a third embodiment of the present disclosure will be described with reference to FIG. 18. Note that the same components as those in the above first and second embodiments are labeled with the same references, and the description thereof will be omitted or simplified.

The rotation drive apparatus 10 according to the above-described embodiments can be used for rotation equipment utilizing the rotation of the rotor 110. In the present embodiment, an X-ray computed tomography (CT) apparatus will be described as an example of the rotation equipment using the rotation drive apparatus 10 according to the above-described embodiments.

FIG. 18 is a side view illustrating an X-ray CT apparatus 600 in which the rotation drive apparatus 10 according to the first embodiment is used. FIG. 18 corresponds to the side view of the rotation drive apparatus 10 according to the first embodiment illustrated in FIG. 1B.

As illustrated in FIG. 18, the X-ray CT apparatus 600 includes the rotation drive apparatus 10 according to the first embodiment, a plate-like part 602, an X-ray irradiation unit 604, an X-ray detection unit 606, a signal amplification unit 608, a cooling unit 610, power supply units 612 and 614, and a power supply control unit 616. The rotation drive apparatus 10 is installed on a mounting (not illustrated). The plate-like part 602, the X-ray irradiation unit 604, the X-ray detection unit 606, the signal amplification unit 608, the cooling unit 610, the power supply units 612 and 614, and the power supply control unit 616 are components installed in the rotor 110 of the rotation drive apparatus 10, respectively.

The plate-like part 602 is a circular plate-like member installed in the cylindrical rotor 110 along the XY-plane. In the center of the plate-like part 602, an imaging port 618 for inserting a subject such as a human or other organism, or the like as an object of X-ray CT is provided. The imaging port 618 is a circular opening centered on the Z-axis which is the rotation axis of the rotor 110. The plate-like part 602 rotates in the Wz direction along with the rotation of the rotor 110 in the Wz direction with the Z-axis as the rotation axis.

Although the manner in which the subject is inserted into the inside of the imaging port 618 is not particularly limited, for example, the subject is inserted into the inside of the imaging port 618 by sliding a bed (not illustrated) on which the subject lies sideways along the Z direction in the Z direction into the inside of the imaging port 618. The subject is inserted into the inside of the imaging port 618 and arranged inside the rotor 110. Note that the subject is not necessarily limited to a living organism such as a human being or the like, but may be an article such as an industrial product.

The X-ray irradiation unit 604, the X-ray detection unit 606, the signal amplification unit 608, the cooling unit 610, the power supply units 612 and 614, and the power supply control unit 616 are mounted on one surface of the plate-like part 602 in the Z direction. The X-ray irradiation unit 604, the X-ray detection unit 606, the signal amplification unit 608, the cooling unit 610, the power supply units 612 and 614, and the power supply control unit 616 rotate in the Wz direction with the Z-axis as the rotation axis by the rotation of the plate-like part 602 accompanied with the rotation of the rotor 110 by the rotation drive apparatus 10. Note that the respective units may be mounted on one or the other surfaces or both surfaces of the plate-like part 602 in the Z direction. The respective units may be arranged between the plurality of coil lines.

The X-ray irradiation unit 604 and the X-ray detection unit 606 are arranged on the same surface of the plate-like part 602 in the Z direction. The X-ray irradiation unit 604 and the X-ray detection unit 606 are arranged so as to face each other in the diameter direction of the annular plate-like part 602 across the Z-axis which is the rotation axis thereof.

The X-ray irradiation unit 604 is an irradiation unit that irradiates the subject with X-rays which are radiation while the rotor 110 rotates to make one rotation around the subject arranged inside the rotor 110. The X-ray irradiation unit 604 is, for example, an X-ray tube. The power supply units 612 and 614 are power supply units that supply a voltage for X-ray irradiation such as a tube voltage to the X-ray tube or the like to the X-ray irradiation unit 604. The power supply control unit 616 is a control device that controls supply of the tube voltage by the power supply units 612 and 614. The cooling unit 610 is a cooling device that cools the X-ray irradiation unit 604, which generates heat during the X-ray irradiation.

The X-ray detection unit 606 is a detector that detects the X-ray irradiated from the X-ray irradiation unit 604 toward the subject and transmitted through the subject. The X-ray detection unit 606 outputs a detection signal corresponding to the detected X-ray. The signal amplification unit 608 amplifies the detection signal output from the X-ray detection unit 606. The signal amplification unit 608 outputs the amplified detection signal to a system control unit (not illustrated). The system control unit generates an X-ray CT image by image processing or the like based on the detection signal from the signal amplification unit 608.

As described above, in the X-ray CT apparatus 600, the rotation drive apparatus 10 can be used as an apparatus that rotates the X-ray irradiation unit 604, the X-ray detection unit 606, and the like by rotating the rotor 110.

Note that, although the X-ray CT apparatus 600 using X-ray has been described in the present embodiment, it is possible to configure a CT apparatus using radiation such as gamma ray instead of X-ray. In this case, the irradiation unit corresponding to the type of the radiation can be used instead of the X-ray irradiation unit 604, and the detection unit corresponding to the type of the radiation can be used instead of the X-ray detection unit 606.

Note that the rotation drive apparatus 10 according to the second embodiment can also be used for the X-ray CT apparatus.

### Modification Embodiment

The present disclosure is not limited to the above embodiments, and various modifications are possible.

For example, in the above embodiments, the cases in which the first and second permanent magnets 101a and 101b are used as the first magnetic force units of the rotor 110, and the first and second coils 201a and 201b are used as the second magnetic force units of the stator 200 are described, but the first magnetic force units and the second magnetic force units are not limited these. Coils may be used as the first magnetic force units of the rotor 110, and permanent magnets or coils may be used as the second magnetic force units of the stator 200. In this case, the current control system 300 may control the drive of the rotor 110 in the same manner as in the above embodiments by controlling the current applied to the coils of the rotor 110 or the stator 200.

In the above embodiment, the X-ray CT apparatus 600 has been described as an example of the rotation equipment in which the rotation drive apparatus 10 is used, but the rotation equipment is not limited thereto. As the rotation equipment in which the rotation drive apparatus 10 is used, any apparatus other than the X-ray CT apparatus 600 can be configured. Further, according to the type of the rotation equipment, components installed in the rotor 110 and rotating together with the rotor 110 can be designed.

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

According to the present disclosure, the rotor can be levitated stably while avoiding enlargement of the whole apparatus.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2024-078798, filed May 14, 2024, which is hereby incorporated by reference herein in its entirety.

What is claimed is:

1. A rotation drive apparatus comprising:
a rotor that includes a first magnetic force unit and is rotatable in a rotation direction around a rotation axis intersecting a direction of gravity;
a stator that includes a second magnetic force unit;
a support that supports the rotor positioned in a first position; and
a control unit,
wherein the first magnetic force unit is arranged circumferentially along the rotation direction on an outer peripheral side surface of the rotor,
wherein the second magnetic force unit is arranged in an arc shape along the rotation direction above the rotor, and
wherein, when the rotor is positioned at the first position and a levitation force with respect to the rotor is less than a predetermined value, the control unit rotates the rotor to a second position where the levitation force is equal to or greater than the predetermined value and levitates the rotor from the second position.

2. The rotation drive apparatus according to claim 1, wherein the control unit levitates the rotor from the first position when the rotor is positioned at the first position and the levitation force is equal to or greater than the predetermined value.

3. The rotation drive apparatus according to claim 1,
wherein the second magnetic force unit includes a plurality of coils, and
wherein the plurality of coils are arranged in line symmetry with respect to an axis along the direction of gravity in a plan view viewed in a direction along the rotation axis.

4. The rotation drive apparatus according to claim 1,
wherein the first magnetic force unit includes a plurality of permanent magnets, and
wherein, in the second position, the plurality of permanent magnets are arranged in line symmetry with respect to an axis along the direction of gravity in a plan view viewed in a direction along the rotation axis.

5. The rotation drive apparatus according to claim 4, wherein, in the second position, a magnetic pole direction of one of the plurality of permanent magnets is parallel to the direction of gravity.

6. The rotation drive apparatus according to claim 4,
wherein the plurality of permanent magnets includes a first magnet group having magnetic poles aligned alternately in the rotation direction and a second magnet group having magnetic poles aligned alternately in a direction intersecting the rotation direction, and
wherein, in the second position, the second magnet group is arranged in line symmetry with respect to an axis along the direction of gravity.

7. The rotation drive apparatus according to claim 1, wherein the levitation force changes with an electric angle of the rotor.

8. The rotation drive apparatus according to claim 1, wherein, when the rotor is supported by the support, the control unit rotates the rotor to a position where a levitation force with respect to the rotor is equal to or greater than a predetermined value, and then makes the rotor supported by the support and stopped.

9. A rotation drive apparatus comprising:
a rotor that includes a first magnetic force unit and is rotatable in a rotation direction around a rotation axis intersecting a direction of gravity;
a stator that includes a second magnetic force unit;
a support that supports the rotor positioned in a first position; and
a control unit,
wherein the first magnetic force unit is arranged circumferentially along the rotation direction on an outer peripheral side surface of the rotor,
wherein the second magnetic force unit is arranged in an arc shape along the rotation direction above the rotor, and
wherein, when the rotor is positioned at the first position and a cogging torque with respect to the rotor is equal to or more than a predetermined value, the control unit rotates the rotor to a second position where the cogging torque is less than the predetermined value and levitates the rotor from the second position.

10. The rotation drive apparatus according to claim 9, wherein the control unit levitates the rotor from the first position when the rotor is positioned at the first position and the cogging torque is less than the predetermined value.

11. The rotation drive apparatus according to claim 9,
wherein the second magnetic force unit includes a plurality of coils, and
wherein the plurality of coils are arranged asymmetrically with respect to an axis along the direction of gravity in a plan view viewed in a direction along the rotation axis.

12. The rotation drive apparatus according to claim 11,
wherein a void is formed between the coils adjacent to each other in the plurality of coils,
wherein the first magnetic force unit includes a plurality of permanent magnets, and
wherein, in the second position, the plurality of permanent magnets are arranged so that one of the permanent magnets does not face both the coil adjacent to the void and the void.

13. The rotation drive apparatus according to claim 11,
wherein the first magnetic force unit includes a plurality of permanent magnets, and
wherein, in the second position, a torque in the rotation direction generated between the plurality of permanent magnets and the plurality of coils is cancelled out.

14. The rotation drive apparatus according to claim 9, wherein the cogging torque changes with an electric angle of the rotor.

15. The rotation drive apparatus according to claim 9, wherein, when the rotor is supported by the support, the control unit rotates the rotor to a position where a cogging torque with respect to the rotor is less than a predetermined value, and then makes the rotor supported by the support and stopped.

16. A control method of a rotation drive apparatus comprising: a rotor that includes a first magnetic force unit and is rotatable in a rotation direction around a rotation axis intersecting a direction of gravity; a stator that includes a second magnetic force unit; and a support that supports the rotor positioned in a first position, wherein the first magnetic force unit is arranged circumferentially along the rotation direction on an outer peripheral side surface of the rotor, and wherein the second magnetic force unit is arranged in an arc shape along the rotation direction above the rotor, the control method comprising:
when the rotor is positioned at the first position and a levitation force with respect to the rotor is less than a predetermined value, rotating the rotor to a second position where the levitation force is equal to or greater than the predetermined value and levitating the rotor from the second position.

17. A control method of a rotation drive apparatus comprising: a rotor that includes a first magnetic force unit and is rotatable in a rotation direction around a rotation axis intersecting a direction of gravity; a stator that includes a second magnetic force unit; and a support that supports the rotor positioned in a first position, wherein the first magnetic force unit is arranged circumferentially along the rotation direction on an outer peripheral side surface of the rotor, and wherein the second magnetic force unit is arranged in an arc shape along the rotation direction above the rotor, the control method comprising:
when the rotor is positioned at the first position and a cogging torque with respect to the rotor is equal to or more than a predetermined value, rotating the rotor to a second position where the cogging torque is less than the predetermined value and levitating the rotor from the second position.

18. Rotation equipment comprising:
a rotation drive apparatus comprising:
a rotor that includes a first magnetic force unit and is rotatable in a rotation direction around a rotation axis intersecting a direction of gravity;
a stator that includes a second magnetic force unit;
a support that supports the rotor positioned in a first position; and
a control unit,
wherein the first magnetic force unit is arranged circumferentially along the rotation direction on an outer peripheral side surface of the rotor,
wherein the second magnetic force unit is arranged in an arc shape along the rotation direction above the rotor, and
wherein, when the rotor is positioned at the first position and a levitation force with respect to the rotor is less than a predetermined value, the control unit rotates the rotor to a second position where the levitation force is equal to or greater than the predetermined value and levitates the rotor from the second position; and
a component installed in the rotor.

19. The rotation equipment according to claim 18, wherein the component includes:
an irradiation unit that irradiates a subject with radiation, the subject being arranged inside the rotor; and
a detection unit that is arranged to face the irradiation unit across the rotation axis and detects the radiation transmitted through the subject.
